(19) United States
(12) Patent Application Publication
(10) Pub. No.: US 2015/0111758 A1
(43) Pub. Date: Apr. 23, 2015

(54) GENE SIGNATURES ASSOCIATED WITH EFFICACY OF POSTMASTECTOMY RADIOTHERAPY IN BREAST CANCER

(71) Applicants: Oslo Universitetssykehus HF, Oslo (NO); Aarhus University, Aarhus C (DK)

(72) Inventors: Therese Sorlie, Oslo (NO); Arnoldo Frigessi, Oslo (NO); Anne-Lise Borresen-Dale, Oslo (NO); Simen Myhre, Oslo (NO); Hayat Mohammed, Oslo (NO); Jens Overgaard, Aarhus C (DK); Jan Alsner, Hojbjerg (DK); Trine Tramm, Aarhus N (DK)

(21) Appl. No.: 14/382,491

(22) PCT Filed: Mar. 6, 2013
(86) PCT No.: PCT/IB2013/001032

§ 371 Date: Sep. 2, 2014

Related U.S. Application Data
(60) Provisional application No. 61/607,316, filed on Mar. 6, 2012.

(51) Int. Cl. C12Q 1/68

(52) U.S. Cl. CPC C12Q 1/6886; C12Q 2600/158; C12Q 2600/106; C12Q 2600/118
USPC 506/2; 435/6.11; 435/6.12; 506/9; 506/16; 435/7.23; 435/7.4; 435/7.92; 436/501; 435/7.1; 506/18; 435/7.94; 435/7.93

(57) ABSTRACT

The present invention relates to compositions, kits, and methods for providing a prognosis and/or determining a treatment course of action in a subject diagnosed with breast cancer. In particular, the present invention relates to gene expression signatures useful in the prognosis, diagnosis, and treatment of breast cancer.

### FIELD OF THE INVENTION

The present invention relates to compositions, kits, and methods for providing a prognosis and/or determining a treatment course of action in a subject diagnosed with breast cancer. In particular, the present invention relates to gene expression signatures useful in the prognosis, diagnosis, and treatment of breast cancer.

### BACKGROUND OF THE INVENTION

Radiotherapy (RT) is known to prevent loco-regional recurrence (LRR), to favour disease free survival and a long-term improvement on overall survival in high-risk patients suffering from breast cancer [1]. RT is the standard treatment of choice after breast conserving surgery, and recommendations for postmastectomy radiotherapy (PMRT) is well established in patients estimated to have a high risk of local regional recurrence (LRR) (e.g. tumor size >5 cm, or involvement of ≧4 lymph nodes) [2]. For patients with a low risk of LRR, treated with mastectomy, recommendations has for a long time been no RT. The reason for this choice is the general assumption that the survival benefit of PMRT is directly proportional to the reduction in LRR, and hence that RT is only beneficial in patients with high risk of LRR. However, large randomized trials exploring the indications for PMRT to high-risk patients have indicated a substantial overall survival benefit after PMRT also in patients with low risk of LRR, e.g. in patients with involvement of 1-3 positive nodes [3,4]. After several studies [8,9,10,11,12] on the largest of these cohorts, the DBCG82 cohort, the positive effect of RT is, however, still speculated to be heterogeneous, and it would be desirable, if a more refined partitioning of patients likely to benefit from RT could be established.

Growing evidence gives reason to believe that genetic studies can improve our knowledge in this direction [13,14,15]. At present, estimation of risk of recurrence and subsequent allocation to adjuvant treatment, including RT, is performed though the combination of clinical and pathological parameters and no validated biomarker predicting RT response is standardized and applicable for daily clinical use [13]. Some of the biological factors reported to be directly related to RT resistance mechanisms include micro-environmental influences as e.g. hypoxia, and the resistance in hypoxic tumors towards treatment such as RT is well known and well described. In 2006 Chi et al. [14] published a “Hypoxia-profile”, able to divide breast cancers into two groups of either high or low expression of the hypoxia response genes. They found that the patients assigned to the high expression group had a significantly lower overall survival and relapse-free survival. The role of estrogen receptor (ER) and human epidermal growth factor receptor 2 (HER2) as predictive biomarkers in combined-modality therapy including RT have also been discussed, and ER-positive tumors has been suggested to represent a more radiosensitive phenotype than ER-negative tumors.

Methods for determining if a subject will benefit from radiation therapy are needed.

### SUMMARY OF THE INVENTION

The present invention relates to compositions, kits, and methods for providing a prognosis and/or determining a treatment course of action in a subject diagnosed with breast cancer. In particular, the present invention relates to gene expression signatures useful in the prognosis, diagnosis, and treatment of breast cancer.

For example, in some embodiments, the present invention provides a method of characterizing breast cancer in a subject diagnosed with breast cancer, comprising: a) measuring the level of expression of one or more (e.g., all) genes (e.g., HLA-DQA, RGS1, DNALI1, IGKC, ADH1B, hCG2023290 or OR8G2) in a sample from a subject diagnosed with breast cancer; and b) characterizing breast cancer based on the level of expression. In some embodiments, the characterizing comprises determining an increased risk of local recurrence, an increased risk of distant metastasis, or determining a treatment course of action (e.g., administration of post mastectomy radiation) based on the level of expression. In some embodiments, an increased level of expression of HLA-DQA, RGS1, DNALI1 and a decreased level of expression of IGKC, ADH1B and OR8G2 is associated with an increased risk of local recurrence of breast cancer. In some embodiments, the treatment course of action is based on a CSVI score calculated from said expression levels, wherein said

$${CVSI}_{i} = \hat{α} \text{?} + {\sum }_{\text{?}} \text{?} X \text{?} . \\ \text{?} \text{indicates text missing or illegible when filed}$$

For example, in some embodiments, a negative CVSI score is indicative of a positive response to post mastectomy radiation and a positive score is indicative of a negative response to post mastectomy radiation. In some embodiments, decreased expression of HLA-DQA, RGS1, DNALI1 and hCG2023290 and increased expression of IGKC, ADH1B and OR8G2 is associated with an increased benefit of radiation therapy. In some embodiments, the characterizing comprises determining a risk of a distant metastasis. For example, in some embodiments, altered expression of IGKC, RGS1 or DNALI1 is associated with increased risk of distant metastasis is the subject. In some embodiments, the sample is a biopsy sample. In some embodiments, expression levels of nucleic acids (e.g., mRNA) or polypeptides of the genes is determined. In some embodiments, expression levels are determined using, for example, microarray analysis, reverse transcriptase PCR, quantitative reverse transcriptase PCR, or hybridization analysis.

In some embodiments, the present invention provides methods of providing a prognosis for a subject with breast cancer, selecting a subject with breast cancer for treatment with a particular therapy, determining an increased risk of local recurrence in a subject with breast cancer, or determining an increased risk of distant metastasis in a subject with breast cancer comprising: a) detecting the level of expression of one or more genes (i.e., 1, 2, 2 or more, 3, 3 or more, 4, 4 or more, 5, five or more, 6, 6 or more, 7, 7 or more, 8, 8 or more, 9, 9 or more, 10, 10 or more, 11, 11 or more, 12, 12 or more, 13, 13 or more, or 14) selected from the group consisting of HLA-DQA, RGS1, DNALI1, IGKC, ADH1B, hCG2023290, OR8G2, C3orf29, ZCCHC17, RTCD1, VANGL1, DERP6, FLJ37970, and RAF1 in a sample from a subject diagnosed with breast cancer; and b) comparing the expression of the one or more genes with a reference expression level for the one or more genes, wherein an altered level of expression relative to the reference provides an indication selected from the group consisting of the likelihood of disease free survival, the likelihood of local recurrence, the likelihood of distant metastasis, and an indication that the subject is a candidate for treatment with a particular therapy.

In some embodiments, the detecting the level of expression of one or more genes comprises determining an expression profile for one or more genes selected from the group consisting of HLA-DQA, RGS1, DNALI1, IGKC, ADH1B, hCG2023290, and OR8G2. In some embodiments, an increased level of expression of HLA-DQA, RGS1, DNALI1 and a decreased level of expression of IGKC, ADH1B and OR8G2 is associated with an increased risk of local recurrence of breast cancer. In some embodiments, the detecting the level of expression of one or more genes comprises determining an expression profile for one or more genes selected from the group consisting of C3orf29, ZCCHC17, RTCD1, VANGL1, DERP6, FLJ37970, and RAF1. In some embodiments, an increased level of expression of one or more of genes selected from the group consisting of C3orf29, ZCCHC17, RTCD1, VANGL1, DERP6, FLJ37970, and RAF1 is associated with an increased risk of local recurrence of breast cancer. In some embodiments, the methods further comprise the step of determining a treatment course of action based on the level of expression. In some embodiments, the treatment course of action is administration of post mastectomy radiation. In some embodiments, the treatment course of action is based on a CSVI score calculated from the expression levels, wherein the

$${CVSI}_{i} = \hat{α} \text{?} + {\sum }_{\text{?}} \text{?} X \text{?} . \\ \text{?} \text{indicates text missing or illegible when filed}$$

In some embodiments, a negative CVSI score is indicative of a positive response to the post mastectomy radiation and a positive score is indicative of a negative response to the post mastectomy radiation. In some embodiments, decreased expression of HLA-DQA, RGS1, DNALI1 and hCG2023290 and increased expression of IGKC, ADH1B and OR8G2 is associated with an increased benefit of radiation therapy. In some embodiments, altered expression of one or more genes selected from the group consisting of IGKC, RGS1 and DNALI1 is associated with increased risk of distant metastasis in the subject.

In some embodiments, the sample is a biopsy sample. In some embodiments, the detecting comprises contacting a sample from the subject with at least informative reagent specific for the one or more genes. In some embodiments, the detecting an expression level comprises detecting the level of nucleic acid of the genes in the sample. In some embodiments, the detecting the level of nucleic acid of the genes in the sample comprises detecting the level of mRNA of the genes in the sample. In some embodiments, the detecting the level of expression of the genes comprises a detection technique selected from the group consisting of microarray analysis, reverse transcriptase PCR, quantitative reverse transcriptase PCR, and hybridization analysis. In some embodiments, the detecting an expression level comprises detecting the level of polypeptide expression from the genes in the sample.

Further embodiments of the present invention provide a method of characterizing breast cancer in a subject diagnosed with breast cancer, comprising: a) measuring the level of expression of SCGB2A1 and/or SCGB1D2 in a sample from a subject diagnosed with breast cancer; and b) characterizing breast cancer based on the level of expression. In some embodiments, the characterizing comprises determining risk of distant metastasis in said subject. In some embodiments, an increased level of expression of the genes is associated with an increased risk of distant metastasis in the subject.

Additional embodiments of the present invention provide the use of detecting the level of expression of HLA-DQA, RGS1, DNALI1, IGKC, ADH1B, hCG2023290, OR8G2, SCGB2A1 or SCGB1D2 in characterizing breast cancer in a subject diagnosed with breast cancer.

Further embodiments provide a kit for characterizing breast cancer in a subject, comprising reagents useful, sufficient, or necessary for detecting and/or characterizing level, presence, or frequency of expression of HLA-DQA, RGS1, DNALI1, IGKC, ADH1B, hCG2023290, OR8G2, SCGB2A1 or SCGB1D2.

The present invention also provides a system comprising a computer readable medium comprising instructions for utilizing information on the level, presence, or frequency of expression of HLA-DQA, RGS1, DNALI1, IGKC, ADH1B, hCG2023290, OR8G2, SCGB2A1 or SCGB1D2 to provide an indication selected from, for example, likelihood of local recurrence of breast cancer, likelihood of distant metastasis of breast cancer, or likelihood of positive response to post mastectomy radiation therapy.

In some embodiments, the present invention provides for use of informative reagents for detecting the level of expression of one or more genes selected from the group consisting of HLA-DQA, RGS1, DNALI1, IGKC, ADH1B, hCG2023290, OR8G2, C3orf29, ZCCHC17, RTCD1, VANGL1, DERP6, FLJ37970, RAF1, SCGB2A1 and SCGB1D2 in characterizing breast cancer in a subject diagnosed with breast cancer. In some embodiments, the characterizing comprises determining an increased risk of local recurrence. In some embodiments, the characterizing comprises determining an increased risk of distant metastasis. In some embodiments, the characterizing comprises determining a treatment course of action. In some embodiments, the treatment course of action comprises post-mastectomy radiation.

In some embodiments, the present invention provides a kit for characterizing breast cancer in a subject, the kit comprising informative reagents useful, sufficient, or necessary for detecting and/or characterizing level, presence, or frequency of expression of one or more genes selected from the group consisting of HLA-DQA, RGS1, DNALI1, IGKC, ADH1B, hCG2023290, OR8G2, C3orf29, ZCCHC17, RTCD1, VANGL1, DERP6, FLJ37970, RAF1, SCGB2A1 and SCGB1D2.

Additional embodiments will be apparent to persons skilled in the relevant art based on the teachings contained herein.

### DESCRIPTION OF THE FIGURES

FIG. 1: Flowchart of the analyses. Upper left panel: pre-selection of candidate genes; set I interaction genes and set J main effect genes. Upper right panel: final selection of interaction genes and bottom panel: validation of the selected interaction via cross validation, classification and prediction.

FIG. 2: The individual interaction effect of each gene in 17. Plots show histograms of the expression (left axis) and the gene-RT-interaction relative risk

$$RR = exp ( X \text{?} )$$

$$\text{?} \text{indicates text missing or illegible when filed}$$

(right axis) for the 17 genes.

FIG. 3: Histogram of the cross-validated reduced score index

$${CVSI}_{i} = \hat{α} \text{?} + {\sum }_{\text{?}} \text{?} X \text{?} . \\ \text{?} \text{indicates text missing or illegible when filed}$$

for all women in the cohort. The score is negative for about 95% of the patients implying that the 7 gene RT-interaction signature introduces an additional, individual specific decrease in the hazard of LRR when radiotherapy is administrated, with respect to the common baseline a. Seven women experienced a reduced benefit of RT, with respect to the baseline.

FIG. 4: Upper two panels show histograms of the CVSI for the subset of women who did not receive RT (left) and those who did (right). These two cohorts are divided into two groups, according to their CVSI being below or above the median CVSI for the specific cohort. The low score group consists of women with CVSI<median (CVSI) and the high score CVSI≧median (CVSI). In red the women with highest CVSI; in blue the women with lowest CVSI, those who would benefit most of RT. In the lower panels the corresponding Kaplan-Meier plots of the LRR-free survival. The difference between low and high CVSI is significant within the no-RT group (log-rank test, p-values given in the figure) but not significant in the RT cohort. The number of patients in each subgroup are: no-RT, low CVSI: 49; no-RT, high CVSI: 51; RT, low CVSI: 47; RT, high CVSI: 48.

FIG. 5: 134 low index patients with positive lymph nodes stratified according to nodal status and randomization. Results show benefit of PMRT for all low index patients regardless of nodal status.

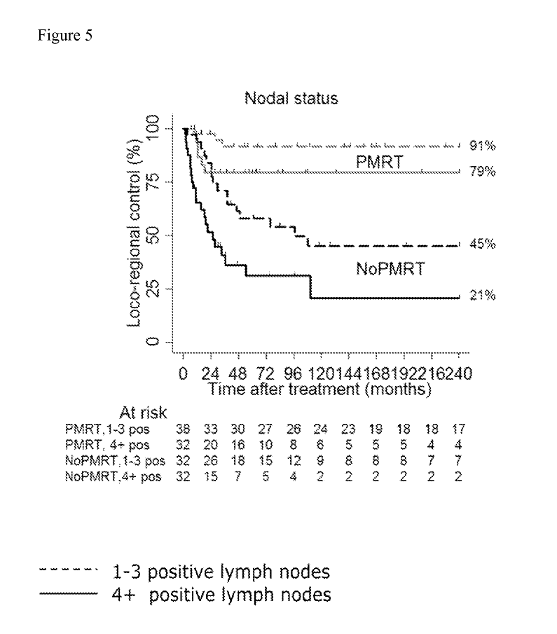

FIG. 6: Validation of signature in new preparation type and on new platform. 150 patients from the original cohort were re-analyzed using either the original data from frozen material and using the array technology (A) or using a different sample type from the same tumours (formalin-fixed paraffin-embedded, FFPE) and using a different technology (qRT-PCR) (B). Identical predictive impact of the signature was observed.

FIG. 7: Validation of signature in independent patient cohort. 116 patients from an independent cohort were analyzed using FFPE material and qRT-PCR. Predictive impact of the signature was confirmed.

FIG. 8: A pair of KM plots for the selected interaction genes were C3orf29, F1137970, VANGL1, DERP6, RTCD1. The p-value for the lower 75% group was 2.40*10̂−8 and the p-value for the upper 25% group is 0.9876.

### DEFINITIONS

To facilitate an understanding of the present invention, a number of terms and phrases are defined below:

As used herein, the terms “detect”, “detecting” or “detection” may describe either the general act of discovering or discerning or the specific observation of a detectably labeled composition.

As used herein, the term “subject” refers to any organisms that are screened using the diagnostic methods described herein. Such organisms preferably include, but are not limited to, mammals (e.g., murines, simians, equines, bovines, porcines, canines, felines, and the like), and most preferably includes humans.

The term “diagnosed,” as used herein, refers to the recognition of a disease by its signs and symptoms, or genetic analysis, pathological analysis, histological analysis, and the like.

As used herein, the term “characterizing cancer in a subject” refers to the identification of one or more properties of a cancer sample in a subject, including but not limited to, the presence of benign, pre-cancerous or cancerous tissue, the stage of the cancer, and the subject's prognosis. Cancers may be characterized by the identification of the expression of one or more cancer marker genes, including but not limited to, those disclosed herein.

As used herein, the term “characterizing breast tissue in a subject” refers to the identification of one or more properties of a breast tissue sample (e.g., including but not limited to, the presence of cancerous tissue, the presence or absence of cancer markers described herein, the presence of pre-cancerous tissue that is likely to become cancerous, and the presence of cancerous tissue that is likely to metastasize). In some embodiments, tissues are characterized by the identification of the expression of one or more cancer marker genes, including but not limited to, the cancer markers disclosed herein.

As used herein, the term “stage of cancer” refers to a qualitative or quantitative assessment of the level of advancement of a cancer. Criteria used to determine the stage of a cancer include, but are not limited to, the size of the tumor and the extent of metastases (e.g., localized or distant).

The term “neoplasm” as used herein refers to any new and abnormal growth of tissue. Thus, a neoplasm can be a premalignant neoplasm or a malignant neoplasm. The term “neoplasm-specific marker” or “breast cancer marker” refers to any biological material that can be used to indicate the presence or characteristics of a neoplasm (e.g., breast cancer). Examples of biological materials include, without limitation, nucleic acids, polypeptides, carbohydrates, fatty acids, cellular components (e.g., cell membranes and mitochondria), and whole cells.

As used herein, the term “metastasis” is meant to refer to the process in which cancer cells originating in one organ or part of the body relocate to another part of the body and continue to replicate. Metastasized cells subsequently form tumors which may further metastasize. Metastasis thus refers to the spread of cancer from the part of the body where it originally occurs to other parts of the body.

As used herein, the term “nucleic acid molecule” refers to any nucleic acid containing molecule, including but not limited to, DNA or RNA. The term encompasses sequences that include any of the known base analogs of DNA and RNA including, but not limited to, 4-acetylcytosine, 8-hydroxy-N6-methyladenosine, aziridinylcytosine, pseudoisocytosine, 5-(carboxyhydroxylmethyl) uracil, 5-fluorouracil, 5-bromouracil, 5-carboxymethylaminomethyl-2-thiouracil, 5-carboxymethylaminomethyluracil, dihydrouracil, 8linic8, N6-isopentenyladenine, 1-methyladenine, 1-methylpseudouracil, 1-methylguanine, 1-methylinosine, 2,2-dimethylguanine, 2-methyladenine, 2-methylguanine, 3-methylcytosine, 5-methylcytosine, N6-methyladenine, 7-methylguanine, 5-methylaminomethyluracil, 5-methoxyaminomethyl-2-thiouracil, beta-D-mannosylqueosine, 5′-methoxycarbonylmethyluracil, 5-methoxyuracil, 2-methylthio-N6-isopentenyladenine, uracil-5-oxyacetic acid methylester, uracil-5-oxyacetic acid, oxybutoxosine, pseudouracil, queosine, 2-thiocytosine, 5-methyl-2-thiouracil, 2-thiouracil, 4-thiouracil, 5-methyluracil, N-uracil-5-oxyacetic acid methylester, uracil-5-oxyacetic acid, pseudouracil, queosine, 2-thiocytosine, and 2,6-diaminopurine.

The term “gene” refers to a nucleic acid (e.g., DNA) sequence that comprises coding sequences necessary for the production of a polypeptide, precursor, or RNA (e.g., rRNA, tRNA). The polypeptide can be encoded by a full length coding sequence or by any portion of the coding sequence so long as the desired activity or functional properties (e.g., enzymatic activity, ligand binding, signal transduction, immunogenicity, etc.) of the full-length or fragments are retained. The term also encompasses the coding region of a structural gene and the sequences located adjacent to the coding region on both the 5′ and 3′ ends for a distance of about 1 kb or more on either end such that the gene corresponds to the length of the full-length mRNA. Sequences located 5′ of the coding region and present on the mRNA are referred to as 5′ non-translated sequences. Sequences located 3′ or downstream of the coding region and present on the mRNA are referred to as 3′ non-translated sequences. The term “gene” encompasses both cDNA and genomic forms of a gene. A genomic form or clone of a gene contains the coding region interrupted with non-coding sequences termed “introns” or “intervening regions” or “intervening sequences.” Introns are segments of a gene that are transcribed into nuclear RNA (hnRNA); introns may contain regulatory elements such as enhancers. Introns are removed or “spliced out” from the nuclear or primary transcript; introns therefore are absent in the messenger RNA (mRNA) transcript. The mRNA functions during translation to specify the sequence or order of amino acids in a nascent polypeptide.

As used herein, the term “oligonucleotide,” refers to a short length of single-stranded polynucleotide chain. Oligonucleotides are typically less than 200 residues long (e.g., between 15 and 100), however, as used herein, the term is also intended to encompass longer polynucleotide chains. Oligonucleotides are often referred to by their length. For example a 24 residue oligonucleotide is referred to as a “24-mer”. Oligonucleotides can form secondary and tertiary structures by self-hybridizing or by hybridizing to other polynucleotides. Such structures can include, but are not limited to, duplexes, hairpins, cruciforms, bends, and triplexes.

As used herein, the terms “complementary” or “complementarity” are used in reference to polynucleotides (i.e., a sequence of nucleotides) related by the base-pairing rules. For example, the sequence “5′-A-G-T-3′,” is complementary to the sequence “3′-T-C-A-S′.” Complementarity may be “partial,” in which only some of the nucleic acids' bases are matched according to the base pairing rules. Or, there may be “complete” or “total” complementarity between the nucleic acids. The degree of complementarity between nucleic acid strands has significant effects on the efficiency and strength of hybridization between nucleic acid strands. This is of particular importance in amplification reactions, as well as detection methods that depend upon binding between nucleic acids.

The term “homology” refers to a degree of complementarity. There may be partial homology or complete homology (i.e., identity). A partially complementary sequence is a nucleic acid molecule that at least partially inhibits a completely complementary nucleic acid molecule from hybridizing to a target nucleic acid is “substantially homologous.” The inhibition of hybridization of the completely complementary sequence to the target sequence may be examined using a hybridization assay (Southern or Northern blot, solution hybridization and the like) under conditions of low stringency. A substantially homologous sequence or probe will compete for and inhibit the binding (i.e., the hybridization) of a completely homologous nucleic acid molecule to a target under conditions of low stringency. This is not to say that conditions of low stringency are such that non-specific binding is permitted; low stringency conditions require that the binding of two sequences to one another be a specific (i.e., selective) interaction. The absence of non-specific binding may be tested by the use of a second target that is substantially non-complementary (e.g., less than about 30% identity); in the absence of non-specific binding the probe will not hybridize to the second non-complementary target.

As used herein, the term “hybridization” is used in reference to the pairing of complementary nucleic acids. Hybridization and the strength of hybridization (i.e., the strength of the association between the nucleic acids) is impacted by such factors as the degree of complementary between the nucleic acids, stringency of the conditions involved, the Tm of the formed hybrid, and the G:C ratio within the nucleic acids. A single molecule that contains pairing of complementary nucleic acids within its structure is said to be “self-hybridized.”

As used herein the term “stringency” is used in reference to the conditions of temperature, ionic strength, and the presence of other compounds such as organic solvents, under which nucleic acid hybridizations are conducted. Under “low stringency conditions” a nucleic acid sequence of interest will hybridize to its exact complement, sequences with single base mismatches, closely related sequences (e.g., sequences with 90% or greater homology), and sequences having only partial homology (e.g., sequences with 50-90% homology). Under ‘medium stringency conditions,” a nucleic acid sequence of interest will hybridize only to its exact complement, sequences with single base mismatches, and closely relation sequences (e.g., 90% or greater homology). Under “high stringency conditions,” a nucleic acid sequence of interest will hybridize only to its exact complement, and (depending on conditions such a temperature) sequences with single base mismatches. In other words, under conditions of high stringency the temperature can be raised so as to exclude hybridization to sequences with single base mismatches.

The term “isolated” when used in relation to a nucleic acid, as in “an isolated oligonucleotide” or “isolated polynucleotide” refers to a nucleic acid sequence that is identified and separated from at least one component or contaminant with which it is ordinarily associated in its natural source. Isolated nucleic acid is such present in a form or setting that is different from that in which it is found in nature. In contrast, non-isolated nucleic acids as nucleic acids such as DNA and RNA found in the state they exist in nature. For example, a given DNA sequence (e.g., a gene) is found on the host cell chromosome in proximity to neighboring genes; RNA sequences, such as a specific mRNA sequence encoding a specific protein, are found in the cell as a mixture with numerous other mRNAs that encode a multitude of proteins. However, isolated nucleic acid encoding a given protein includes, by way of example, such nucleic acid in cells ordinarily expressing the given protein where the nucleic acid is in a chromosomal location different from that of natural cells, or is otherwise flanked by a different nucleic acid sequence than that found in nature. The isolated nucleic acid, oligonucleotide, or polynucleotide may be present in single-stranded or double-stranded form. When an isolated nucleic acid, oligonucleotide or polynucleotide is to be utilized to express a protein, the oligonucleotide or polynucleotide will contain at a minimum the sense or coding strand (i.e., the oligonucleotide or polynucleotide may be single-stranded), but may contain both the sense and anti-sense strands (i.e., the oligonucleotide or polynucleotide may be double-stranded).

As used herein, the term “purified” or “to purify” refers to the removal of components (e.g., contaminants) from a sample. For example, antibodies are purified by removal of contaminating non-immunoglobulin proteins; they are also purified by the removal of immunoglobulin that does not bind to the target molecule. The removal of non-immunoglobulin proteins and/or the removal of immunoglobulins that do not bind to the target molecule results in an increase in the percent of target-reactive immunoglobulins in the sample. In another example, recombinant polypeptides are expressed in bacterial host cells and the polypeptides are purified by the removal of host cell proteins; the percent of recombinant polypeptides is thereby increased in the sample.

As used herein, the term “sample” is used in its broadest sense. In one sense, it is meant to include a specimen or culture obtained from any source, as well as biological and environmental samples. Biological samples may be obtained from animals (including humans) and encompass fluids, solids, tissues, and gases. Biological samples include blood products, such as plasma, serum and the like. Such examples are not however to be construed as limiting the sample types applicable to the present invention.

### DETAILED DESCRIPTION OF THE INVENTION

The present invention relates to compositions, kits, and methods for providing a prognosis and/or determining a treatment course of action in a subject diagnosed with breast cancer. In particular, the present invention relates to gene expression signatures useful in the prognosis, diagnosis, and treatment of breast cancer.

Many studies have identified gene signatures for prediction of survival after breast cancer. However, few have focused on deriving gene expression profiles associated with effect of radiation on disease-free survival. Here, we present a statistical framework to explore gene-radiotherapy interaction effects on local recurrence (LRR) and distance metastasis (DM) free-survival. To select genes with potential predictive value of the efficacy of radiation on the risk of relapse we studied the interaction between gene expressions and radiotherapy (RT) in a Cox proportional hazards model with L1 penalty. A two-stage selection procedure was implemented to enable detection of second order effects. A first set of genes influencing the risk of LRR via interaction with RT were identified (i.e., the following genes with corresponding exemplary mRNA, cDNA or protein sequences: HLA-DQA, e.g., GenBank Access. No. NM_{—}002122.3), RGS1 (e.g., GenBank Access. No. NM_{—}002922), DNALI1 (e.g., Genbank Access. No. NM_{—}012144.2), IGKC (e.g., Genbank Access. No. AF113889.1), ADH1B (e.g., Genbank Access. No. NM_{—}000668.4), hCG2023290 (e.g., Genbank Access. No. EAW76729; protein sequence), and OR8G2 (e.g., Genbank Access. No. NM_{—}001007249.1).

Increasing expression level of HLA-DQA, RGS1, DNALI1 and a fourth gene in chromosome 7 resulted in higher risk of LRR, while higher expression of genes IGKC, ADH1B and OR8G2 decreases the risk when RT is given to patients. A CVSI index combining the effect of the 7 genes was defined to predict the benefit of radiation. It indicated an individual specific differential benefit from RT in addition to an overall improvement in LRR-free survival. Within the No-RT cohort, women with low CVSI, i.e., those who would have benefited most of radiotherapy, had a significantly worse survival than those with high CVSI. SCGB2A1 and SCGB1D2 appeared to also influence the risk of DM through their interaction with radiotherapy. Further, IGCK and RGS1 were found to be associated with DM-free survival in the 1-3 nodal group. While in the 4+ nodal group, only DNALI1 had a significant effect on the risk of DM.

Identifying gene expression based predictive markers is difficult much due to the interaction between therapy-related improvement of outcome and true prognosis. For breast cancer, some studies have focused on identifying gene signatures conferring resistance to therapy while others have attempted to identify signatures associated with sensitivity to treatment. Few studies have investigated the ability of gene expression patterns to predict response to radiotherapy. Experiments conducted during the course of development of embodiments of the present invention identified genes whose expressions interact with RT and thereby influence the risk of loco-regional recurrence and/or distant metastasis. To this aim, a double selection procedure was implemented. The pre-selection of candidate genes was done by fitting a multivariate Cox model with 17910 gene expressions and their second order interactions with radiotherapy. The Lasso shrinkage method was utilized to handle the high-dimensionality of the predictors. The candidate genes were identified by varying the Lasso penalty weight and picking all the genes that were selected at the various levels of penalization. This approach has an advantage over a univariate selection of genes that does not take into account correlation between genes. None of the clinical covariates were included at the pre-selection stage, in order to maximize chances for genes to show strong interaction with RT. Nothing was optimized in the first stage, so there was no need to include this choice in a cross-validation loop. Next the 206 genes obtained in this manner were regressed in another multivariate Cox model adjusting this time for known clinical prognostic factors including radiotherapy. A parsimonious model was found by 5-fold cross validation. Finally, this double selection procedure identified seven genes for which the hazard of LRR changed when their expression level varied when RT was administered. Furthermore, two genes influencing DM-free survival by interacting with radiation were also identified in a similar analysis.

A cross-validated score index (CVSI), calculated for the combined effect of the seven genes, indicated the existence of an individual specific reduction in risk of LRR if RT is given. This finds use as a means of predicting the individual benefit of radiation for a woman given her tumor's expression profile of these seven genes.

The gene major histocompatibility complex, class II, DQ alpha 1 (HLA-DQA1) is positioned on chromosome arm 6p21.3. It has been reported that a lack of this gene was associated with breast cancer in southern Taiwanese women[30]. However, it is mostly reported in relation to type 1 diabetes. The immunoglobulin kappa constant (IGKC) is found on chromosome arm 2p12 encoding for the kappa light chain of immunoglobulins. The gene has never been reported with a relation to breast cancer. However, it has been reported in relation to B cell malignancies [31]. The regulator of G-protein signalling 1 (RGS1) gene is positioned on chromosome arm 1q31. The gene has been associated with multiple sclerosis [32], and melanoma [33] but never breast cancer. The expression of the RGS1 gene has been studied in normal and cancer cells exposed to gamma-radiation[34].

The alcohol dehydrogenase IB (class I), beta polypeptide (ADH1B) resides on chromosome arm 4q21-q23. Genetic polymorphisms of the gene have been studied in relation to alcohol consumption and risk of breast cancer [35] [36] but no effect of the polymorphism was found. The gene dynein, axonemal, light intermediate polypeptide 1 (DNALI1) resides on chromosome arm 1p35.1. Downregulation of the DNALI has been reported in more malignant tumors in diploid breast carcinoma [37]. The olfactory receptor, family 8, subfamily G, member 2 (OR8G2) resides on chromosome arm 11q24. The gene encodes a 7-transmembrane G-protein coupled receptor (GPCR). The gene has not been reported in relation to any cancers. As for the last gene-probe identified, the transcript was not found in any genes. A BLAST of the sequence matched to a sequence on chromosome arm 7.

SCGB2A1 (GenBank Access. No. NM_{—}002407.2) and SCGB1D1 (e.g., GenBank Access. No. NM_{—}006552.1) are the secretoglobin, family 2A, and secretoglobin, family 1D, member 2, respectively. They are positioned in close vicinity on chromosome arm 11q13, a genomic region that was amplified in 40% of the samples. The SCGB2A1 is also known as mammaglobin and is a marker for disseminating tumor cells (DTC) in breast cancer [38] [39] [40]. It has also been proposed as a prognostic marker in ovarian cancer [41] and a novel serum marker in breast cancer [42]. SCGB1D1 has been found as a heterodimer in human tears [43]. The expression of these two genes was likely to be copy number driven, and probably highly correlated due to their neighboring genomic positions.

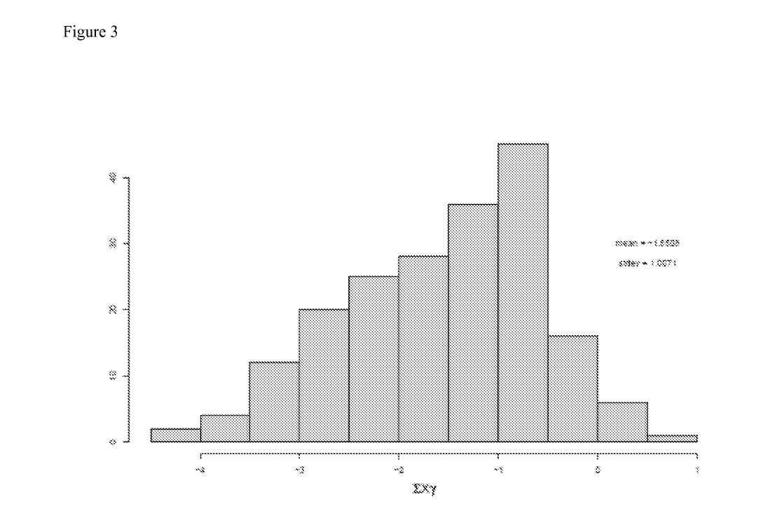

The data presented herein demonstrates that there is a significant difference in LRR-free survival between the patients with high CVSI score and those with a low CVSI score for the no-RT group (FIG. 3C), showing a differential response to RT. In fact, women with low CVSI appear to have a much worse LRR-free survival and identify a group of patients who would have benefited most from RT. Within the RT-group no significant difference in the LRR-free survival between low and high CVSI patients was found (FIG. 3D). Since the overall benefit of RT (RR=0.2634) in terms of reducing the risk of LRR is much larger than the marginal interaction effect of the 7 genes, the individual specific reduction or increase in risk for all the women is outweighted, including those (%5) women with extreme expression values. In addition, for 95% of the patients in this study the expression level of these 7 genes contributed to a further reduction of the risk if RT is given. Several studies on the DBCG cohort have shown a positive effect of RT on the total population; fewer LRR and/or DM after 18 years follow-up and the benefit was similar in patients with 1-3 and more than 4 positive nodes [4, 10].

The present invention discloses that tumor size and number of positive lymph nodes were significantly associated with the LRR risk, together with the 46 main effect and 7 RT-interaction genes; the menopausal status and the ER status do not appear as significant. It is known that the prognostic value of ER status levels out after 5 years, and decreases with longer follow-up time. The interaction of expression of these 7 genes with RT has a differential effect on the risk of LRR; for four of them, HLA-DQA, RGS1, DNALI1 and hCG2023290, the relative risk of LRR increases in conjunction with radiotherapy when the expression increases. In other words, higher expressions of these genes in the tumor, indicates limited effect of radiotherapy on reducing LRR. For IGKC, ADH1B and OR8G2 the opposite is observed; benefit of RT on risk of LRR when expression is high.

Jointly, the expressions of the seven genes interact with radiotherapy protectively, reducing the risk of LRR for 95% of the women. Our index CVSI allows scoring women according to their expected benefit from radiotherapy, compared to the overall benefit (represented by the population baseline). It is based on a measure of the expression of the 7 genes from tumor tissue. All women benefit from radiotherapy, but some can be predicted to benefit significantly more than average. The fact that one may identify those who may have an additive beneficial effect of the radiation compared with the overall benefit provides valuable clinical information.

When considering only women who did not undergo radiotherapy, the group with sub-median CVSI, who would have benefit most of radiotherapy, had a significantly worse survival than the group of women with high CVSI, who would have benefit less of radiotherapy. The difference disappeared in the group of women who did undergo radiotherapy. This shows that CVSI has the capacity to identify women who would benefit of radiotherapy beyond average.

When the outcome was changed to the risk of distant metastasis (DM), two genes (SCGB2A1 and SCGB1D2) showed an interaction with radiotherapy. For these genes the risk of DM increases with the expression level when undergoing radiotherapy. In routinely pathological practice mammoglobin is used as an IHC stain to help determine whether metastatic cells originate from the breast or elsewhere, and mammoglobin is found in the normal breast luminal cells. Lower expression in the tumor cells indicates a loss of differentiation, meaning that the tissue has a lesser degree of resemblance with the normal tissue, and hence a higher malignancy-potential. Mammaglobin B has been related to metastasis in breast cancer.

We found that among the 7 genes, with a significant interaction with radiotherapy in affecting LRR risk, IGCK and RGS1 are also associated with DM free survival, but only in the group of women with few (1, 2 or 3) positive lymph nodes. On the contrary, for women with more positive lymph nodes (4 or more), only DNALI1 was identified.

An additional gene signature was identified following reanalysis of the data. This gene set comprises C3orf29 (e.g., GenBank Access. No. AM393050.1), ZCCHC17 (e.g., GenBank Access. No. NM_{—}016505.2), RTCD1 (e.g., GenBank Access. No. JF432517.1), VANGL1 (e.g., GenBank Access. No. Accession: NM_{—}138959.2), DERP6 (e.g., GenBank Access. No. AB013910.1), FLJ37970 (e.g., GenBank Access. No. AK095289), and RAF1 (e.g., GenBank Access. No. BC018119.2). In some preferred embodiments, increased expression of one or more (i.e., 1, 2, 3, 4, 5, 6, or 7) of these genes as compared to a reference is indicative of an increased risk of local recurrence (LRR) or distance metastasis (DM) free-survival. Analysis of expression of one or more genes from this gene signature may be combined with analysis of expression of one or more genes from the first gene signature (i.e., HLA-DQA, RGS1, DNALI1, IGKC, ADH1B, hCG2023290 and OR8G2).

Embodiments of the present invention provide research, diagnostic, prognostic, predictive and therapeutic kits, systems, methods and uses for characterizing breast cancer. For example, embodiments of the present invention provide a gene expression signature (e.g., one or more (e.g., 1, 2, 3, 4, 5, 6 or 7) of HLA-DQA, RGS1, DNALI1, IGKC, ADH1B, hCG2023290 and OR8G2 and/or one or more (e.g., 1, 2, 3, 4, 5, 6 or 7) of C3orf29, ZCCHC17, RTCD1, VANGL1, DERP6, FLJ37970, and RAF1, and associated CVSI indexes that identify women likely to benefit from post-mastectomy radiation. For example, in some embodiments, lower expression of HLA-DQA, RGS1, DNALI1 and hCG2023290 and higher expression of IGKC, ADH1B and OR8G2 is associated with an increased benefit of radiation therapy.

Further embodiments of the present invention provide compositions and methods for predicting risk of distant metastasis in breast cancer patients. For example, in some embodiments, altered expression of SCGB2A1 and SCGB1D2 is associated with increased risk of distant metastasis.

Gene expression data for patients may be obtained using any suitable methods, including but not limited to, those disclosed herein.

Any patient sample containing breast tumor may be tested according to methods of embodiments of the present invention. By way of non-limiting examples, the sample may be tissue (e.g., a biopsy sample), blood, breast milk, or a fraction thereof (e.g., plasma, serum).

The expression levels of breast cancer marker genes are detected using a variety of nucleic acid and protein detection techniques known to those of ordinary skill in the art, including but not limited to: nucleic acid sequencing; nucleic acid hybridization; nucleic acid amplification; and protein detection.

### I. DNA and RNA Detection—Breast Cancer Gene Expression Signature (BCGES) Informative Reagents

The gene products of the present invention are detected using a variety of nucleic acid techniques known to those of ordinary skill in the art, including but not limited to: nucleic acid sequencing; nucleic acid hybridization; and, nucleic acid amplification. In particular, the gene products are detected with BCGES informative reagents specific for the gene products of one or more of the following genes: HLA-DQA, RGS1, DNALI1, IGKC, ADH1B, hCG2023290, OR8G2, C3orf29, ZCCHC17, RTCD1, VANGL1, DERP6, FLJ37970, RAF1, SCGB2A1 and SCGB1D2. Thus the BCGES informative reagents may comprise reagents such as primers and probes for detection of the gene products by sequencing, hybridization, amplification, microarray analysis, and related methodologies.

1. Sequencing

Illustrative non-limiting examples of nucleic acid sequencing techniques include, but are not limited to, chain terminator (Sanger) sequencing and dye terminator sequencing. Those of ordinary skill in the art will recognize that because RNA is less stable in the cell and more prone to nuclease attack experimentally RNA is usually reverse transcribed to DNA before sequencing.

Chain terminator sequencing uses sequence-specific termination of a DNA synthesis reaction using modified nucleotide substrates. Extension is initiated at a specific site on the template DNA by using a short radioactive, or other labeled, oligonucleotide primer complementary to the template at that region. The oligonucleotide primer is extended using a DNA polymerase, standard four deoxynucleotide bases, and a low concentration of one chain terminating nucleotide, most commonly a di-deoxynucleotide. This reaction is repeated in four separate tubes with each of the bases taking turns as the di-deoxynucleotide. Limited incorporation of the chain terminating nucleotide by the DNA polymerase results in a series of related DNA fragments that are terminated only at positions where that particular di-deoxynucleotide is used. For each reaction tube, the fragments are size-separated by electrophoresis in a slab polyacrylamide gel or a capillary tube filled with a viscous polymer. The sequence is determined by reading which lane produces a visualized mark from the labeled primer as you scan from the top of the gel to the bottom.

Dye terminator sequencing alternatively labels the terminators. Complete sequencing can be performed in a single reaction by labeling each of the di-deoxynucleotide chain-terminators with a separate fluorescent dye, which fluoresces at a different wavelength.

A variety of nucleic acid sequencing methods are contemplated for use in the methods of the present disclosure including, for example, chain terminator (Sanger) sequencing, dye terminator sequencing, and high-throughput sequencing methods. Many of these sequencing methods are well known in the art. See, e.g., Sanger et al., Proc. Natl. Acad. Sci. USA 74:5463-5467 (1997); Maxam et al., Proc. Natl. Acad. Sci. USA 74:560-564 (1977); Drmanac, et al., Nat. Biotechnol. 16:54-58 (1998); Kato, Int. J. Clin. Exp. Med. 2:193-202 (2009); Ronaghi et al., Anal. Biochem. 242:84-89 (1996); Margulies et al., Nature 437:376-380 (2005); Ruparel et al., Proc. Natl. Acad. Sci. USA 102:5932-5937 (2005), and Harris et al., Science 320:106-109 (2008); Levene et al., Science 299:682-686 (2003); Korlach et al., Proc. Natl. Acad. Sci. USA 105:1176-1181 (2008); Branton et al., Nat. Biotechnol. 26(10):1146-53 (2008); Eid et al., Science 323:133-138 (2009); each of which is herein incorporated by reference in its entirety.

A number of DNA sequencing techniques are known in the art, including fluorescence-based sequencing methodologies (See, e.g., Birren et al., Genome Analysis: Analyzing DNA, 1, Cold Spring Harbor, N.Y.; herein incorporated by reference in its entirety). In some embodiments, automated sequencing techniques understood in that art are utilized. In some embodiments, parallel sequencing of partitioned amplicons (PCT Publication No: WO2006084132 to Kevin McKernan et al., herein incorporated by reference in its entirety) is utilized. In some embodiments, bridge amplification (see, e.g., WO 2000/018957, U.S. Pat. No. 7,972,820; 7,790,418 and Adessi et al., Nucleic Acids Research (2000): 28(20): E87; each of which are herein incorporated by reference) is utilized. In some embodiments, DNA sequencing by parallel oligonucleotide extension (See, e.g., U.S. Pat. No. 5,750,341 to Macevicz et al., and U.S. Pat. No. 6,306,597 to Macevicz et al., both of which are herein incorporated by reference in their entireties) is utilized. Additional examples of sequencing techniques include the Church polony technology (Mitra et al., 2003, Analytical Biochemistry 320, 55-65; Shendure et al., 2005 Science 309, 1728-1732; U.S. Pat. No. 6,432,360, U.S. Pat. No. 6,485,944, U.S. Pat. No. 6,511,803; herein incorporated by reference in their entireties), the 454 picotiter pyrosequencing technology (Margulies et al., 2005 Nature 437, 376-380; US 20050130173; herein incorporated by reference in their entireties), the Solexa single base addition technology (Bennett et al., 2005, Pharmacogenomics, 6, 373-382; U.S. Pat. No. 6,787,308; U.S. Pat. No. 6,833,246; herein incorporated by reference in their entireties), the Lynx massively parallel signature sequencing technology (Brenner et al. (2000). Nat. Biotechnol. 18:630-634; U.S. Pat. No. 5,695,934; U.S. Pat. No. 5,714,330; herein incorporated by reference in their entireties), and the Adessi PCR colony technology (Adessi et al. (2000). Nucleic Acid Res. 28, E87; WO 00018957; herein incorporated by reference in its entirety).

Next-generation sequencing (NGS) methods share the common feature of massively parallel, high-throughput strategies, with the goal of lower costs in comparison to older sequencing methods (see, e.g., Voelkerding et al., Clinical Chem., 55: 641-658, 2009; MacLean et al., Nature Rev. Microbiol., 7: 287-296; each herein incorporated by reference in their entirety). NGS methods can be broadly divided into those that typically use template amplification and those that do not. Amplification-requiring methods include pyrosequencing commercialized by Roche as the 454 technology platforms (e.g., GS 20 and GS FLX), the Solexa platform commercialized by Illumina, and the Supported Oligonucleotide Ligation and Detection (SOLiD) platform commercialized by Applied Biosystems. Non-amplification approaches, also known as single-molecule sequencing, are exemplified by the HeliScope platform commercialized by Helicos BioSciences, and emerging platforms commercialized by VisiGen, Oxford Nanopore Technologies Ltd., Life Technologies/Ion Torrent, and Pacific Biosciences, respectively.

2. Hybridization

Illustrative non-limiting examples of nucleic acid hybridization techniques include, but are not limited to, in situ hybridization (ISH), microarray, and Southern or Northern blot. In situ hybridization (ISH) is a type of hybridization that uses a labeled complementary DNA or RNA strand as a probe to localize a specific DNA or RNA sequence in a portion or section of tissue (in situ), or, if the tissue is small enough, the entire tissue (whole mount ISH). DNA ISH can be used to determine the structure of chromosomes. RNA ISH is used to measure and localize mRNAs and other transcripts (e.g., gene products) within tissue sections or whole mounts. Sample cells and tissues are usually treated to fix the target transcripts in place and to increase access of the probe. The probe hybridizes to the target sequence at elevated temperature, and then the excess probe is washed away. The probe that was labeled with either radio-, fluorescent- or antigen-labeled bases is localized and quantitated in the tissue using either autoradiography, fluorescence microscopy or immunohistochemistry, respectively. ISH can also use two or more probes, labeled with radioactivity or the other non-radioactive labels, to simultaneously detect two or more transcripts.

In some embodiments, gene products are detected using fluorescence in situ hybridization (FISH). In some embodiments, FISH assays utilize bacterial artificial chromosomes (BACs). These have been used extensively in the human genome sequencing project (see Nature 409: 953-958 (2001)) and clones containing specific BACs are available through distributors that can be located through many sources, e.g., NCBI. Each BAC clone from the human genome has been given a reference name that unambiguously identifies it. These names can be used to find a corresponding GenBank sequence and to order copies of the clone from a distributor.

The present invention further provides a method of performing a FISH assay on human prostate cells, human prostate tissue or on the fluid surrounding said human prostate cells or human prostate tissue. Specific protocols are well known in the art and can be readily adapted for the present invention. Guidance regarding methodology may be obtained from many references including: In situ Hybridization: Medical Applications (eds. G. R. Coulton and J. de Belleroche), Kluwer Academic Publishers, Boston (1992); In situ Hybridization: In Neurobiology; Advances in Methodology (eds. J. H. Eberwine, K. L. Valentino, and J. D. Barchas), Oxford University Press Inc., England (1994); In situ Hybridization: A Practical Approach (ed. D. G. Wilkinson), Oxford University Press Inc., England (1992)); Kuo, et al., Am. J. Hum. Genet. 49:112-119 (1991); Klinger, et al., Am. J. Hum. Genet. 51:55-65 (1992); and Ward, et al., Am. J. Hum. Genet. 52:854-865 (1993)). There are also kits that are commercially available and that provide protocols for performing FISH assays (available from e.g., Oncor, Inc., Gaithersburg, Md.). Patents providing guidance on methodology include U.S. Pat. No. 5,225,326; 5,545,524; 6,121,489 and 6,573,043. All of these references are hereby incorporated by reference in their entirety and may be used along with similar references in the art and with the information provided in the Examples section herein to establish procedural steps convenient for a particular laboratory.

In some embodiments, the present invention utilizes nuclease protection assays. Nuclease protection assays are useful for identification of one or more RNA molecules of known sequence even at low total concentration. The extracted RNA is first mixed with antisense RNA or DNA probes that are complementary to the sequence or sequences of interest and the complementary strands are hybridized to form double-stranded RNA (or a DNA-RNA hybrid). The mixture is then exposed to ribonucleases that specifically cleave only single-stranded RNA but have no activity against double-stranded RNA. When the reaction runs to completion, susceptible RNA regions are degraded to very short oligomers or to individual nucleotides; the surviving RNA fragments are those that were complementary to the added antisense strand and thus contained the sequence of interest. Suitable nuclease protection assays, include, but are not limited to those described in U.S. Pat. No. 5,770,370; EP 2290101A3; US 20080076121; US 20110104693; each of which is incorporated herein by reference in its entirety. In some embodiments, the present invention utilizes the quantitative nuclease protection assay provided by HTG Molecular Diagnostics, Inc. (Tucson, Ariz.).

3. Microarrays

Different kinds of biological assays are called microarrays including, but not limited to: DNA microarrays (e.g., cDNA microarrays and oligonucleotide microarrays); protein microarrays; tissue microarrays; transfection or cell microarrays; chemical compound microarrays; and, antibody microarrays. A DNA microarray, commonly known as gene chip, DNA chip, or biochip, is a collection of microscopic DNA spots attached to a solid surface (e.g., glass, plastic or silicon chip) forming an array for the purpose of expression profiling or monitoring expression levels for thousands of genes simultaneously. The affixed DNA segments are known as probes, thousands of which can be used in a single DNA microarray. Microarrays can be used to identify disease genes or transcripts (e.g., gene products) by comparing gene expression in disease and normal cells. Microarrays can be fabricated using a variety of technologies, including but not limiting: printing with fine-pointed pins onto glass slides; photolithography using pre-made masks; photolithography using dynamic micromirror devices; ink jetprinting; or, electrochemistry on microelectrode arrays.

Southern and Northern blotting is used to detect specific DNA or RNA sequences, respectively. DNA or RNA extracted from a sample is fragmented, electrophoretically separated on a matrix gel, and transferred to a membrane filter. The filter bound DNA or RNA is subject to hybridization with a labeled probe complementary to the sequence of interest. Hybridized probe bound to the filter is detected. A variant of the procedure is the reverse Northern blot, in which the substrate nucleic acid that is affixed to the membrane is a collection of isolated DNA fragments and the probe is RNA extracted from a tissue and labeled.

In some embodiments, the present invention utilizes digital molecular barcoding technology, preferably in conjunction with an nCounter Analysis System (Nanostring Technologies, Seattle, Wash.) for the detection of gene expression products. This technique utilizes a digital color-coded barcode technology that is based on direct multiplexed measurement of gene expression and offers high levels of precision and sensitivity (>1 copy per cell). The technology uses molecular “barcodes” and single molecule imaging to detect and count hundreds of unique transcripts in a single reaction. Each color-coded barcode is attached to a single target-specific probe corresponding to a gene of interest. Mixed together with controls, they form a multiplexed CodeSet. Each color-coded barcode represents a single target molecule. Barcodes hybridize directly to the target molecules and can be individually counted. In preferred embodiments, a hybridization step employs two ˜50 base probes (the capture and reporter probes) per mRNA that hybridize in solution. The reporter probe carries the barcode signal; the capture probe allows the complex to be immobilized for data collection. After hybridization, the excess probes are removed and the probe/target complexes aligned and immobilized in an nCounter Cartridge. Sample cartridges are placed in a digital analyzer for data collection. Color codes on the surface of the cartridge are counted and tabulated for each target molecule. See e.g., U.S. Pat. Publ. 20100015607, 20100047924; and 20100112710; each of which is incorporated by reference herein in its entirety.

4. Amplification

Nucleic acids (e.g., gene products) may be amplified prior to or simultaneous with detection. Illustrative non-limiting examples of nucleic acid amplification techniques include, but are not limited to, polymerase chain reaction (PCR), reverse transcription polymerase chain reaction (RT-PCR), transcription-mediated amplification (TMA), ligase chain reaction (LCR), strand displacement amplification (SDA), and nucleic acid sequence based amplification (NASBA). Those of ordinary skill in the art will recognize that certain amplification techniques (e.g., PCR) require that RNA be reversed transcribed to DNA prior to amplification (e.g., RT-PCR), whereas other amplification techniques directly amplify RNA (e.g., TMA and NASBA).

The polymerase chain reaction (U.S. Pat. Nos. 4,683,195, 4,683,202, 4,800,159 and 4,965,188, each of which is herein incorporated by reference in its entirety), commonly referred to as PCR, uses multiple cycles of denaturation, annealing of primer pairs to opposite strands, and primer extension to exponentially increase copy numbers of a target nucleic acid sequence. In a variation called RT-PCR, reverse transcriptase (RT) is used to make a complementary DNA (cDNA) from mRNA, and the cDNA is then amplified by PCR to produce multiple copies of DNA. For other various permutations of PCR see, e.g., U.S. Pat. Nos. 4,683,195, 4,683,202 and 4,800,159; Mullis et al., Meth. Enzymol. 155: 335 (1987); and, Murakawa et al., DNA 7: 287 (1988), each of which is herein incorporated by reference in its entirety.

Transcription mediated amplification (U.S. Pat. Nos. 5,480,784 and 5,399,491, each of which is herein incorporated by reference in its entirety), commonly referred to as TMA, synthesizes multiple copies of a target nucleic acid sequence autocatalytically under conditions of substantially constant temperature, ionic strength, and pH in which multiple RNA copies of the target sequence autocatalytically generate additional copies. See, e.g., U.S. Pat. Nos. 5,399,491 and 5,824,518, each of which is herein incorporated by reference in its entirety. In a variation described in U.S. Publ. No. 20060046265 (herein incorporated by reference in its entirety), TMA optionally incorporates the use of blocking moieties, terminating moieties, and other modifying moieties to improve TMA process sensitivity and accuracy.

The ligase chain reaction (Weiss, R., Science 254: 1292 (1991), herein incorporated by reference in its entirety), commonly referred to as LCR, uses two sets of complementary DNA oligonucleotides that hybridize to adjacent regions of the target nucleic acid. The DNA oligonucleotides are covalently linked by a DNA ligase in repeated cycles of thermal denaturation, hybridization and ligation to produce a detectable double-stranded ligated oligonucleotide product.

Strand displacement amplification (Walker, G. et al., Proc. Natl. Acad. Sci. USA 89: 392-396 (1992); U.S. Pat. Nos. 5,270,184 and 5,455,166, each of which is herein incorporated by reference in its entirety), commonly referred to as SDA, uses cycles of annealing pairs of primer sequences to opposite strands of a target sequence, primer extension in the presence of a dNTPaS to produce a duplex hemiphosphorothioated primer extension product, endonuclease-mediated nicking of a hemimodified restriction endonuclease recognition site, and polymerase-mediated primer extension from the 3′ end of the nick to displace an existing strand and produce a strand for the next round of primer annealing, nicking and strand displacement, resulting in geometric amplification of product. Thermophilic SDA (tSDA) uses thermophilic endonucleases and polymerases at higher temperatures in essentially the same method (EP Pat. No. 0 684 315).

Other amplification methods include, for example: nucleic acid sequence based amplification (U.S. Pat. No. 5,130,238, herein incorporated by reference in its entirety), commonly referred to as NASBA; one that uses an RNA replicase to amplify the probe molecule itself (Lizardi et al., BioTechnol. 6: 1197 (1988), herein incorporated by reference in its entirety), commonly referred to as Qβ replicase; a transcription based amplification method (Kwoh et al., Proc. Natl. Acad. Sci. USA 86:1173 (1989)); and, self-sustained sequence replication (Guatelli et al., Proc. Natl. Acad. Sci. USA 87: 1874 (1990), each of which is herein incorporated by reference in its entirety). For further discussion of known amplification methods see Persing, David H., “In Vitro Nucleic Acid Amplification Techniques” in Diagnostic Medical Microbiology: Principles and Applications (Persing et al., Eds.), pp. 51-87 (American Society for Microbiology, Washington, D.C. (1993)).

In some embodiments, the present invention utilizes multiplexed amplification and detection techniques. See, e.g., Wong et al., Biotechniques (2005) 39(1):1-11; and Bustin, J. Mol. Endocrinol. (2000) 25: 169-193; each of which is incorporated by reference herein in its entirety. Suitable multiplexed amplification-based detection techniques include, but are not limited to, the hybridization probe four oligonucleotide method, the hybridization probe three oligonucleotide method, and methods utilizing hydrolysis probes (two primers and one specific probe per target molecule), molecular beacons (two primers and one specific probe per target molecule), scorpions, sunrise primers (two PCR primers per target molecule), and LUX primers (two PCR primer per target molecule). Another suitable multiplexed, amplification-based technique is the ICEP1ex/STAR technology system from PrimeraDX (Mansfield, Mass.). This technique utilizes end-labeled PCR for amplification of specific target molecules followed by detection by real time sampling via capillary electrophoresis. See e.g., U.S. Pat. Publ. 20100221725; 20110300537; and 20120100600; each of which is incorporated by reference herein in its entirety.

5. Detection Methods

Non-amplified or amplified nucleic acids can be detected by any conventional means. For example, the gene products can be detected by hybridization with a detectably labeled probe and measurement of the resulting hybrids. Illustrative non-limiting examples of detection methods are described below.

One illustrative detection method, the Hybridization Protection Assay (HPA) involves hybridizing a chemiluminescent oligonucleotide probe (e.g., an acridinium ester-labeled (AE) probe) to the target sequence, selectively hydrolyzing the chemiluminescent label present on unhybridized probe, and measuring the chemiluminescence produced from the remaining probe in a luminometer. See, e.g., U.S. Pat. No. 5,283,174 and Norman C. Nelson et al., Nonisotopic Probing, Blotting, and Sequencing, ch. 17 (Larry J. Kricka ed., 2d ed. 1995, each of which is herein incorporated by reference in its entirety).

Another illustrative detection method provides for quantitative evaluation of the amplification process in real-time. Evaluation of an amplification process in “real-time” involves determining the amount of amplicon in the reaction mixture either continuously or periodically during the amplification reaction, and using the determined values to calculate the amount of target sequence initially present in the sample. A variety of methods for determining the amount of initial target sequence present in a sample based on real-time amplification are well known in the art. These include methods disclosed in U.S. Pat. Nos. 6,303,305 and 6,541,205, each of which is herein incorporated by reference in its entirety. Another method for determining the quantity of target sequence initially present in a sample, but which is not based on a real-time amplification, is disclosed in U.S. Pat. No. 5,710,029, herein incorporated by reference in its entirety.

Amplification products may be detected in real-time through the use of various self-hybridizing probes, most of which have a stem-loop structure. Such self-hybridizing probes are labeled so that they emit differently detectable signals, depending on whether the probes are in a self-hybridized state or an altered state through hybridization to a target sequence. By way of non-limiting example, “molecular torches” are a type of self-hybridizing probe that includes distinct regions of self-complementarity (referred to as “the target binding domain” and “the target closing domain”) which are connected by a joining region (e.g., non-nucleotide linker) and which hybridize to each other under predetermined hybridization assay conditions. In a preferred embodiment, molecular torches contain single-stranded base regions in the target binding domain that are from 1 to about 20 bases in length and are accessible for hybridization to a target sequence present in an amplification reaction under strand displacement conditions. Under strand displacement conditions, hybridization of the two complementary regions, which may be fully or partially complementary, of the molecular torch is favored, except in the presence of the target sequence, which will bind to the single-stranded region present in the target binding domain and displace all or a portion of the target closing domain. The target binding domain and the target closing domain of a molecular torch include a detectable label or a pair of interacting labels (e.g., luminescent/quencher) positioned so that a different signal is produced when the molecular torch is self-hybridized than when the molecular torch is hybridized to the target sequence, thereby permitting detection of probe:target duplexes in a test sample in the presence of unhybridized molecular torches. Molecular torches and a variety of types of interacting label pairs are disclosed in U.S. Pat. No. 6,534,274, herein incorporated by reference in its entirety.

Another example of a detection probe having self-complementarity is a “molecular beacon.” Molecular beacons include nucleic acid molecules having a target complementary sequence, an affinity pair (or nucleic acid arms) holding the probe in a closed conformation in the absence of a target sequence present in an amplification reaction, and a label pair that interacts when the probe is in a closed conformation. Hybridization of the target sequence and the target complementary sequence separates the members of the affinity pair, thereby shifting the probe to an open conformation. The shift to the open conformation is detectable due to reduced interaction of the label pair, which may be, for example, a fluorophore and a quencher (e.g., DABCYL and EDANS). Molecular beacons are disclosed in U.S. Pat. Nos. 5,925,517 and 6,150,097, herein incorporated by reference in its entirety.

Other self-hybridizing probes are well known to those of ordinary skill in the art. By way of non-limiting example, probe binding pairs having interacting labels, such as those disclosed in U.S. Pat. No. 5,928,862 (herein incorporated by reference in its entirety) might be adapted for use in the present invention. Probe systems used to detect single nucleotide polymorphisms (SNPs) might also be utilized in the present invention. Additional detection systems include “molecular switches,” as disclosed in U.S. Publ. No. 20050042638, herein incorporated by reference in its entirety. Other probes, such as those comprising intercalating dyes and/or fluorochromes, are also useful for detection of amplification products in the present invention. See, e.g., U.S. Pat. No. 5,814,447 (herein incorporated by reference in its entirety).

### II. Protein Detection—BCGES Informative Reagents

The gene products of the present invention may further be proteins and be detected using a variety of protein detection techniques known to those of ordinary skill in the art, including but not limited to: sequencing, mass spectrometry and immunoassays. In particular, the gene products are detected with BCGES informative reagents specific for the protein gene products of one or more of the following genes: HLA-DQA, RGS1, DNALI1, IGKC, ADH1B, hCG2023290, OR8G2, C3orf29, ZCCHC17, RTCD1, VANGL1, DERP6, F1137970, RAF1, SCGB2A1 and SCGB1D2. Thus the BCGES informative reagents may comprise reagents such as antibodies (e.g., primary and secondary antibodies) and other protein detection probes.

1. Sequencing

Illustrative non-limiting examples of protein sequencing techniques include, but are not limited to, mass spectrometry and Edman degradation.

Mass spectrometry can, in principle, sequence any size protein but becomes computationally more difficult as size increases. A protein is digested by an endoprotease, and the resulting solution is passed through a high pressure liquid chromatography column. At the end of this column, the solution is sprayed out of a narrow nozzle charged to a high positive potential into the mass spectrometer. The charge on the droplets causes them to fragment until only single ions remain. The peptides are then fragmented and the mass-charge ratios of the fragments measured. The mass spectrum is analyzed by computer and often compared against a database of previously sequenced proteins in order to determine the sequences of the fragments. The process is then repeated with a different digestion enzyme, and the overlaps in sequences are used to construct a sequence for the protein.

In the Edman degradation reaction, the peptide to be sequenced is adsorbed onto a solid surface (e.g., a glass fiber coated with polybrene). The Edman reagent, phenylisothiocyanate (PTC), is added to the adsorbed peptide, together with a mildly basic buffer solution of 12% trimethylamine, and reacts with the amine group of the N-terminal amino acid. The terminal amino acid derivative can then be selectively detached by the addition of anhydrous acid. The derivative isomerizes to give a substituted phenylthiohydantoin, which can be washed off and identified by chromatography, and the cycle can be repeated. The efficiency of each step is about 98%, which allows about 50 amino acids to be reliably determined

2. Immunoassays

Illustrative non-limiting examples of immunoassays include, but are not limited to: immunoprecipitation; Western blot; ELISA; immunohistochemistry; immunocytochemistry; flow cytometry; and, immuno-PCR. Polyclonal or monoclonal antibodies detectably labeled using various techniques known to those of ordinary skill in the art (e.g., colorimetric, fluorescent, chemiluminescent or radioactive) are suitable for use in the immunoassays.

Immunoprecipitation is the technique of precipitating an antigen out of solution using an antibody specific to that antigen. The process can be used to identify protein complexes present in cell extracts by targeting a protein believed to be in the complex. The complexes are brought out of solution by insoluble antibody-binding proteins isolated initially from bacteria, such as Protein A and Protein G. The antibodies can also be coupled to sepharose beads that can easily be isolated out of solution. After washing, the precipitate can be analyzed using mass spectrometry, Western blotting, or any number of other methods for identifying constituents in the complex.

A Western blot, or immunoblot, is a method to detect protein in a given sample of tissue homogenate or extract. It uses gel electrophoresis to separate denatured proteins by mass. The proteins are then transferred out of the gel and onto a membrane, typically polyvinyldiflroride or nitrocellulose, where they are probed using antibodies specific to the protein of interest. As a result, researchers can examine the amount of protein in a given sample and compare levels between several groups.

An ELISA, short for Enzyme-Linked ImmunoSorbent Assay, is a biochemical technique to detect the presence of an antibody or an antigen in a sample. It utilizes a minimum of two antibodies, one of which is specific to the antigen and the other of which is coupled to an enzyme. The second antibody will cause a chromogenic or fluorogenic substrate to produce a signal. Variations of ELISA include sandwich ELISA, competitive ELISA, and ELISPOT. Because the ELISA can be performed to evaluate either the presence of antigen or the presence of antibody in a sample, it is a useful tool both for determining serum antibody concentrations and also for detecting the presence of antigen.

Immunohistochemistry and immunocytochemistry refer to the process of localizing proteins in a tissue section or cell, respectively, via the principle of antigens in tissue or cells binding to their respective antibodies. Visualization is enabled by tagging the antibody with color producing or fluorescent tags. Typical examples of color tags include, but are not limited to, horseradish peroxidase and alkaline phosphatase. Typical examples of fluorophore tags include, but are not limited to, fluorescein isothiocyanate (FITC) or phycoerythrin (PE).

Flow cytometry is a technique for counting, examining and sorting microscopic particles suspended in a stream of fluid. It allows simultaneous multiparametric analysis of the physical and/or chemical characteristics of single cells flowing through an optical/electronic detection apparatus. A beam of light (e.g., a laser) of a single frequency or color is directed onto a hydrodynamically focused stream of fluid. A number of detectors are aimed at the point where the stream passes through the light beam; one in line with the light beam (Forward Scatter or FSC) and several perpendicular to it (Side Scatter (SSC) and one or more fluorescent detectors). Each suspended particle passing through the beam scatters the light in some way, and fluorescent chemicals in the particle may be excited into emitting light at a lower frequency than the light source. The combination of scattered and fluorescent light is picked up by the detectors, and by analyzing fluctuations in brightness at each detector, one for each fluorescent emission peak, it is possible to deduce various facts about the physical and chemical structure of each individual particle. FSC correlates with the cell volume and SSC correlates with the density or inner complexity of the particle (e.g., shape of the nucleus, the amount and type of cytoplasmic granules or the membrane roughness).

Immuno-polymerase chain reaction (IPCR) utilizes nucleic acid amplification techniques to increase signal generation in antibody-based immunoassays. Because no protein equivalence of PCR exists, that is, proteins cannot be replicated in the same manner that nucleic acid is replicated during PCR, the only way to increase detection sensitivity is by signal amplification. The target proteins are bound to antibodies which are directly or indirectly conjugated to oligonucleotides. Unbound antibodies are washed away and the remaining bound antibodies have their oligonucleotides amplified. Protein detection occurs via detection of amplified oligonucleotides using standard nucleic acid detection methods, including real-time methods.

In some embodiments, mass spectrometry is utilized to detect protein gene expression products. Preferred techniques include, but are not limited to, matrix-assisted laser desorption/ionization time of flight (MALDI-TOF MS) and electrospray mass spectrometry (ESMS). See, e.g., Mann et al., Annu. Rev. Biochem (2001) 70:437-73.

### III. Data Analysis

In some embodiments, a computer-based analysis program is used to translate the raw data generated by the detection assay (e.g., the presence, absence, or amount of a given marker or markers) into data of predictive value for a clinician. The clinician can access the predictive data using any suitable means. Thus, in some preferred embodiments, the present invention provides the further benefit that the clinician, who is not likely to be trained in genetics or molecular biology, need not understand the raw data. The data is presented directly to the clinician in its most useful form. The clinician is then able to immediately utilize the information in order to optimize the care of the subject.

The present invention contemplates any method capable of receiving, processing, and transmitting the information to and from laboratories conducting the assays, information provides, medical personal, and subjects. For example, in some embodiments of the present invention, a sample (e.g., a biopsy or a serum or urine sample) is obtained from a subject and submitted to a profiling service (e.g., clinical lab at a medical facility, genomic profiling business, etc.), located in any part of the world (e.g., in a country different than the country where the subject resides or where the information is ultimately used) to generate raw data. Where the sample comprises a tissue or other biological sample, the subject may visit a medical center to have the sample obtained and sent to the profiling center, or subjects may collect the sample themselves (e.g., a urine sample) and directly send it to a profiling center. Where the sample comprises previously determined biological information, the information may be directly sent to the profiling service by the subject (e.g., an information card containing the information may be scanned by a computer and the data transmitted to a computer of the profiling center using an electronic communication systems). Once received by the profiling service, the sample is processed and a profile is produced (i.e., expression data), specific for the diagnostic or prognostic information desired for the subject.

The profile data is then prepared in a format suitable for interpretation by a treating clinician. For example, rather than providing raw expression data, the prepared format may represent a diagnosis or risk assessment (e.g., presence or absence of a pseudogene) for the subject, along with recommendations for particular treatment options. The data may be displayed to the clinician by any suitable method. For example, in some embodiments, the profiling service generates a report that can be printed for the clinician (e.g., at the point of care) or displayed to the clinician on a computer monitor.

In some embodiments, the information is first analyzed at the point of care or at a regional facility. The raw data is then sent to a central processing facility for further analysis and/or to convert the raw data to information useful for a clinician or patient. The central processing facility provides the advantage of privacy (all data is stored in a central facility with uniform security protocols), speed, and uniformity of data analysis. The central processing facility can then control the fate of the data following treatment of the subject. For example, using an electronic communication system, the central facility can provide data to the clinician, the subject, or researchers.

In some embodiments, the subject is able to directly access the data using the electronic communication system. The subject may chose further intervention or counseling based on the results. In some embodiments, the data is used for research use. For example, the data may be used to further optimize the inclusion or elimination of markers as useful indicators of a particular condition or stage of disease or as a companion diagnostic to determine a treatment course of action.

IV. In vivo Imaging

Gene products may also be detected using in vivo imaging techniques, including but not limited to: radionuclide imaging; positron emission tomography (PET); computerized axial tomography, X-ray or magnetic resonance imaging method, fluorescence detection, and chemiluminescent detection. In some embodiments, in vivo imaging techniques are used to visualize the presence of or expression of cancer markers in an animal (e.g., a human or non-human mammal). For example, in some embodiments, cancer marker mRNA or protein is labeled using a labeled antibody specific for the cancer marker. A specifically bound and labeled antibody can be detected in an individual using an in vivo imaging method, including, but not limited to, radionuclide imaging, positron emission tomography, computerized axial tomography, X-ray or magnetic resonance imaging method, fluorescence detection, and chemiluminescent detection. Methods for generating antibodies to the cancer markers of the present invention are described below.

The in vivo imaging methods of embodiments of the present invention are useful in the identification of cancers that express gene products (e.g., prostate cancer). In vivo imaging is used to visualize the presence or level of expression of a ncRNA. Such techniques allow for diagnosis without the use of an unpleasant biopsy. The in vivo imaging methods of embodiments of the present invention can further be used to detect metastatic cancers in other parts of the body.

In some embodiments, reagents (e.g., antibodies) specific for the cancer markers of the present invention are fluorescently labeled. The labeled antibodies are introduced into a subject (e.g., orally or parenterally). Fluorescently labeled antibodies are detected using any suitable method (e.g., using the apparatus described in U.S. Pat. No. 6,198,107, herein incorporated by reference).

In other embodiments, antibodies are radioactively labeled. The use of antibodies for in vivo diagnosis is well known in the art. Sumerdon et al., (Nucl. Med. Biol 17:247-254 have described an optimized antibody-chelator for the radioimmunoscintographic imaging of tumors using Indium-111 as the label. Griffin et al., (J Clin Onc 9:631-640 [1991]) have described the use of this agent in detecting tumors in patients suspected of having recurrent colorectal cancer. The use of similar agents with paramagnetic ions as labels for magnetic resonance imaging is known in the art (Lauffer, Magnetic Resonance in Medicine 22:339-342 [1991]). The label used will depend on the imaging modality chosen. Radioactive labels such as Indium-111, Technetium-99m, or Iodine-131 can be used for planar scans or single photon emission computed tomography (SPECT). Positron emitting labels such as Fluorine-19 can also be used for positron emission tomography (PET). For MRI, paramagnetic ions such as Gadolinium (III) or Manganese (II) can be used.

Radioactive metals with half-lives ranging from 1 hour to 3.5 days are available for conjugation to antibodies, such as scandium-47 (3.5 days) gallium-67 (2.8 days), gallium-68 (68 minutes), technetiium-99m (6 hours), and indium-111 (3.2 days), of which gallium-67, technetium-99m, and indium-111 are preferable for gamma camera imaging, gallium-68 is preferable for positron emission tomography.

A useful method of labeling antibodies with such radiometals is by means of a bifunctional chelating agent, such as diethylenetriaminepentaacetic acid (DTPA), as described, for example, by Khaw et al. (Science 209:295 [1980]) for In-111 and Tc-99m, and by Scheinberg et al. (Science 215:1511 [1982]). Other chelating agents may also be used, but the 1-(p-carboxymethoxybenzyl)EDTA and the carboxycarbonic anhydride of DTPA are advantageous because their use permits conjugation without affecting the antibody's immunoreactivity substantially.

Another method for coupling DPTA to proteins is by use of the cyclic anhydride of DTPA, as described by Hnatowich et al. (Int. J. Appl. Radiat. Isot. 33:327 [1982]) for labeling of albumin with In-111, but which can be adapted for labeling of antibodies. A suitable method of labeling antibodies with Tc-99m which does not use chelation with DPTA is the pretinning method of Crockford et al., (U.S. Pat. No. 4,323,546, herein incorporated by reference).

A method of labeling immunoglobulins with Tc-99m is that described by Wong et al. (Int. J. Appl. Radiat. Isot., 29:251 [1978]) for plasma protein, and recently applied successfully by Wong et al. (J. Nucl. Med., 23:229 [1981]) for labeling antibodies.

In the case of the radiometals conjugated to the specific antibody, it is likewise desirable to introduce as high a proportion of the radiolabel as possible into the antibody molecule without destroying its immunospecificity. A further improvement may be achieved by effecting radiolabeling in the presence of the ncRNA, to insure that the antigen binding site on the antibody will be protected. The antigen is separated after labeling.

In still further embodiments, in vivo biophotonic imaging (Xenogen, Almeda, Calif.) is utilized for in vivo imaging. This real-time in vivo imaging utilizes luciferase. The luciferase gene is incorporated into cells, microorganisms, and animals (e.g., as a fusion protein with a cancer marker of the present invention). When active, it leads to a reaction that emits light. A CCD camera and software is used to capture the image and analyze it.

### V. Compositions & Kits

Compositions for use in the diagnostic methods described herein include, but are not limited to, kits comprising one or more BCGES informative reagents as described above. In some embodiments, the kits comprise one or more BCGES informative reagents for detecting altered gene expression in a sample from a subject having or suspected of having cervical cancer, wherein the reagents are specific detection of one or more gene products from the following genes: HLA-DQA, RGS1, DNALI1, IGKC, ADH1B, hCG2023290, OR8G2, C3orf29, ZCCHC17, RTCD1, VANGL1, DERP6, FLJ37970, RAF1, SCGB2A1 and SCGB1D2.

In some embodiments, the kits contain BCGES informative reagents specific for a cancer gene marker, in addition to detection reagents and buffers. In preferred embodiments, the BCGES informative reagent is a probe(s) that specifically hybridizes to a respective gene product(s) of the one or more genes, a set(s) of primers that amplify a respective gene product(s) of the one or more genes, an antigen binding protein(s) that binds to a respective gene products) of the one or more genes, or a sequencing primer(s) that hybridizes to and allows sequencing of a respective gene products) of the one or more genes. The probe and antibody compositions of the present invention may also be provided in the form of an array. In preferred embodiments, the kits contain all of the components necessary to perform a detection assay, including all controls, directions for performing assays, and any necessary software for analysis and presentation of results.

In some embodiments, the kits include instructions for using the reagents contained in the kit for the detection and characterization of cancer in a sample from a subject. In some embodiments, the instructions further comprise the statement of intended use required by the U.S. Food and Drug Administration (FDA) in labeling in vitro diagnostic products. The FDA classifies in vitro diagnostics as medical devices and requires that they be approved through the 510(k) procedure. Information required in an application under 510(k) includes: 1) The in vitro diagnostic product name, including the trade or proprietary name, the common or usual name, and the classification name of the device; 2) The intended use of the product; 3) The establishment registration number, if applicable, of the owner or operator submitting the 510(k) submission; the class in which the in vitro diagnostic product was placed under section 513 of the FD&C Act, if known, its appropriate panel, or, if the owner or operator determines that the device has not been classified under such section, a statement of that determination and the basis for the determination that the in vitro diagnostic product is not so classified; 4) Proposed labels, labeling and advertisements sufficient to describe the in vitro diagnostic product, its intended use, and directions for use. Where applicable, photographs or engineering drawings should be supplied; 5) A statement indicating that the device is similar to and/or different from other in vitro diagnostic products of comparable type in commercial distribution in the U.S., accompanied by data to support the statement; 6) A 510(k) summary of the safety and effectiveness data upon which the substantial equivalence determination is based; or a statement that the 510(k) safety and effectiveness information supporting the FDA finding of substantial equivalence will be made available to any person within 30 days of a written request; 7) A statement that the submitter believes, to the best of their knowledge, that all data and information submitted in the premarket notification are truthful and accurate and that no material fact has been omitted; 8) Any additional information regarding the in vitro diagnostic product requested that is necessary for the FDA to make a substantial equivalency determination. Additional information is available at the Internet web page of the U.S. FDA.

### EXPERIMENTAL

The following examples are provided in order to demonstrate and further illustrate certain preferred embodiments and aspects of the present invention and are not to be construed as limiting the scope thereof.

### Example 1

### Material and Methods

### The DBCG82bc Cohort

The DBCG82 trial explores the indication for post mastectomy radiotherapy (RT) to high-risk patients. Part of the study included 3083 women surgically treated for high-risk breast cancer (DBCG82bc). After mastectomy, the premenopausal women (DBCG 82 b) were randomized to receive cyclophosphamide, methotrexate and 5-fluorouracil (CMF) chemotherapy (8 cycles)+RT, or CMF only (9 cycles). The postmenopausal women (DBCG 82 c) were randomized to receive either Tamoxifen (30 mg daily for 1 year)+RT, or Tamoxifen only. The addition of PMRT improved overall survival by approximately 10%, and resulted in an 80% reduction of loco-regional recurrences (LRR), with no significant late side-effects [8, 9]. Later studies of the same cohort, with 18 years of follow-up, concluded that fewer patients experienced LRR and/or distant metastasis [10], and a subgroup analysis of 1152 node positive patients showed that the survival benefit from RT was similar in patients with 1-3 and 4 positive nodes, and not strictly associated with the risk of LRR [4]. The positive effect of RT obtained for the total population enrolled in the DBCG82bc cohort was, however, speculated to be heterogeneous, and a subgroup analysis of 1000 patients divided into three prognostic subgroups of LR risk, defined by the combination of various clinico-pathological parameters, showed the largest translation of LRR into reduction of breast cancer mortality within the most favorable prognosis group [11]. An analysis of molecular features of the tumors and the benefit of PMRT [12] of the same 1000 patients, showed constructed subtypes of hormone receptor and HER2 expression to be predictive of LRR and survival after PMRT, and the overall survival benefit most evident for the more favorable luminal subtypes. The two endpoints considered in this study were loco-regional recurrence after mastectomy (without simultaneous distant metastases) and distant metastases. The time to DM was either observed or censored at the last follow-up time. The censoring of the time to LRR incorporated information on DM as the two events are non-independent [4]. The endpoint LRR was registered as observed if it occurred before distance metastases, more than one month after DM or if it was the only event observed (no DM) by the end of the study. The time to LRR was censored; at the time to DM if it happened simultaneously with DM (i.e., one month before or after DM) or if only DM occurred by the end of the follow-up time. Finally the time to LRR was censored at the last follow-up time if neither endpoints were observed. We included 5 clinical prognostic factors: tumor size, with three categories, <20 mm, 21-50 mm, >50 mm; the number of positive lymph nodes, in three categories, 0, 1-3, >4; estrogen receptor status, negative or positive; menopausal status, pre- or post-menopausal; and, radiotherapy recipient, yes or no. See Table 1 for a description of these covariates.

### The Expression Data

The gene expression platform used in this work was the “Applied Biosystem Human Genome Survey Microarray v2.0” (Applied Biosystem, Foster City, US). The microarray contained 29098 gene probes (60-mers) and several control probes monitoring every experimental step in the cRNA amplification, labeling and hybridization procedure [44]. The system utilizes chemiluminescense for capturing gene expression signaling. The probe-to-probe normalization within each array was handled automatically by the AB1700 system. In every spot along-side the 60-mer probes, shorter 20-mer probes were provided, hybridizing with a fluorescently labeled control RNA. The signal from the 20-mer control probes was adjusted to be identical across the entire array, and the chemiluminescense derived signal from each gene probe was adjusted proportionally. The input of total RNA into the one round of amplification and labeling was 500 ng, and 10 μg of labeled and amplified cRNA was hybridized onto the array for 16 hours prior to washing and signal detection. Quality measures of the control probes (low present call or failed amplification efficiency due to poly-a-tail bias) were utilized to exclude 20 arrays from further analyses. For details see [16].

TABLE 1
The clinical factors, and therapy, with the number of patient in
each category. Total cohart in this study included 195 women.

| clinical factors | categories | # of samples |
|---|---|---|
| Radiotherapy | Yes | 100 |
| | No | 95 |
| Menopausal status | Premenopausal | 89 |
| | Postmenopausal | 106 |
| Tumor size | <20 mm | 59 |
| | 20-50 mm | 108 |
| | >50 mm | 28 |
| Number of nodes | negative nodes | 11 |
| | 1-3 positive nodes mm | 102 |
| | 4+ positive nodes | 82 |
| ER status | positive | 144 |
| | negative | 51 |

To identify genes whose expression is associated with the effect of radiotherapy on the risk of locoregional recurrence (LRR), we studied the interaction between gene expressions and radiotherapy (received or not). Interaction effects are weaker than main effects, and difficult to detect: a preselection step was necessary [20], [21]. First, at the pre-selection step, a Cox proportional hazards model with lasso penalty [16, 17] was fitted, with the gene expressions as main effects in addition to their interaction with a binary radiotherapy variable. None of the clinical covariates are included at this stage.

More precisely, let tl, . . . , to be the times to LRR or censored times of the n=195 individuals in the study and let d1, . . . , dn be the corresponding censoring indicators. The Cox model is described by the hazard of LRR at time t for a woman with gene expression values x₁=x_{i,1}, . . . , x_{i,} [symbol] as

$${x}_{1} = \text{?} , … , \text{?} as$$

$$h ( t \vert \text{?} … , \text{?} ) = {h}_{0} ( t ) exp ( {\sum }_{j = 1}^{p} \text{?} {β}_{j} + {\sum }_{g = 1}^{p} {T}_{i} \text{?} {γ}_{g} )$$

$$\text{?} \text{indicates text missing or illegible when filed}$$

where h0(t) denotes the baseline hazard, Ti is the indicator of whether patient I received radiotherapy (Ti=1) or not (Ti=0). The log partial likelihood is given by

$${l}_{p} ( β , γ ) = \text{?} {d}_{i} ( \text{?} β + {T}_{i} \text{?} γ ) - log ( \text{?} exp ( \text{?} β + {T}_{j} \text{?} γ ) , \\ \text{?} \text{indicates text missing or illegible when filed}$$

where xᵢ is the p-dimensional vector of covariates for individual i, β and γ are the vectors of regression parameters. When applying the Lasso shrinkage, the penalized estimates are found by maximizing

$${l}_{p} ( β , γ ) - λ ( {\sum }_{j = 1}^{p} \vert {β}_{j} \vert + {\sum }_{g = 1}^{p} \vert {γ}_{g} \vert )$$

or equivalently maximizing the log partial likelihood

$${l}_{p} ( β , γ ) subject to ( {\sum }_{j = 1}^{p} \vert {β}_{j} \vert + {\sum }_{g = 1}^{p} \vert {γ}_{g} \vert ) < g .$$

Here, λ and s denote the penalization parameters which are in one-to-one correspondence though there is no closed conversion formula. This step of the analysis was carried out using glcoxph library in R [22], which can handle high-dimensional vector of predictors, the covariates were on the same scale. The Lasso procedure selects a number of main effect genes and (RT×expression)-interaction genes for each fixed value of the tuning parameter. Taking the union of all these gene lists, identified at the various level of penalization, two sets were constructed: J, the set of genes with direct main effect on the risk of LRR and I, the set of genes associated to LRR through their interaction with RT. At the pre-selection stage, we varied the Lasso penalty weight s on a grid of 400 values in [0.05, 20] (grid [0.05,5] was suggested in [23]).

Next, the pre-selected genes along with the clinical factors of Table 1 are regressed in a multivariate Cox model. The candidate genes of set J were included as main effects while the interactions between RT and genes of set I were modeled as second order effects. Now the instantaneous risk of LRR at time t for a women with gene expressions xi,1 . . . , xi,p and clinical covariates zi, 1, . . . , zi,5 is modeled as

$$h ( \text{?} \vert {x}_{i} , {z}_{i} ) = {h}_{0} ( t ) exp ( α {T}_{i} + {\sum }_{k = 1}^{4} {θ}_{k} {z}_{k , i} + {\sum }_{j ∈ J} {x}_{i , j} {β}_{j} + \text{?} {T}_{i} {x}_{i , g} {γ}_{g} ) . \\ \text{?} \text{indicates text missing or illegible when filed}$$

Here, α is the effect of RT, Φk the effect of clinical covariate zk, βj and γj the main and interaction effects of gene j. Again, Lasso shrinkage is applied to avoid overfitting and the optimal penalty weight λopt identified via 5-fold cross validation[19]. The final selection of genes associated with RT and influencing the LRR-free survival is done at λopt. This stage of the analysis was carried out using glmpath library in R [24], which allows inclusion of none penalized covariates.

The standard errors of the significant interaction coefficients were estimated via nonparametric 0.632-bootstrap [27]: we sampled m≈0.632n=124 women from the cohort of 195 without replacement; then we estimated the coefficients in the Cox model of the second stage at the optimal cross-validated penalty level λ=λopt. This bootstrap procedure was repeated 200 times and the standard errors estimated from the sampling distributions of the bootstrapped coefficients.

Finally, after a set I* of significant interaction genes was obtained from the double selection procedure a cross-validated score index was computed for each women, from leave-one-out crossvalidation[18]. This approach mimics external validation as the coefficients used in the computation of the score index for woman I are estimated excluding woman i. In practice, for I=1, . . . , n, we (i) removed subject I from data; (ii) Lasso estimates \hat{γ}(−i) of the interaction genes I* are obtained by applying the Cox model of the final selection step to the reduced (n−1 patients) data. (iii) repeated steps (i)-(ii) over all n subjects; (iv) computed the score for each women using the cross-validated estimates as

$${CVSI}_{i} = \hat{α} \text{?} + {\sum }_{\text{?}} \text{?} X \text{?} . \\ \text{?} \text{indicates text missing or illegible when filed}$$

By dividing the n predictive scores at the median, the patients were classified into two groups and their LRR-free survival probabilities compared. The CVSI differentiates between women expected to benefit most from RT (low CVSI) and those who would benefit less (high CVSI) with respect to a baseline.

A further validation of the predictive value of the genes was based on using a different outcome, namely, time to distance metastasis (DM). Excluding 11 women with no positive lymph nodes, the rest of the patients were divided into two nodal groups; 1-3 nodes and 4+ nodes group. A simple multivariate Cox model was fitted for each such sub-group of patients, also adjusting for the clinical factors.

The proportionality assumption for the significant genes and the clinical covariates was checked by visual inspection of the re-scaled Schoenfeld residuals against survival time (with a natural spline smoother) and the goodness-of-fit test as in [25].

### Results

### Selection of Genes

At the pre-selection stage, we identified 206 genes, 178 as main effect and 44 as interaction effect genes, with 16 genes in common Goeman's [26] test for global predictive significance for these 206 selected genes was highly significant (p-value=0.00159), indicating a strong association of these genes with the outcome. While the p-value for the same test on all 17910 genes was 0.0396 and the average p-value for 1000 random subsets of 206 genes was 0.06552 (library globaltest in R). In the subsequent selection stage, 46 main effect genes and 7 interaction genes (denoted as 17) were selected at the optimal level of penalty λopt=4.000265 found via 5-fold cross validation. The 7 interaction genes are reported in Table 2 along with the estimated interaction coefficients, the hazard ratios and the corresponding bootstrapped standard errors. The estimated interaction coefficients are small except for RGS1 (0.2810 se 0.1323) and DNALI1 (0.3763 se 0.1429). Radiotherapy (RR=0.26), the number of positive lymph nodes (RR=1.72) and tumor size (RR=1.12) were also significantly associated with LRR, see Table 3. We evaluated whether the proportional hazards assumption was valid for the seven genes and the selected clinical covariates. Only DNALI1 appeared to have a time dependent effect; increasing up to approximately three months and then decreasing.

### Interaction Effects of the Seven Genes

To assess the effect of each gene in interaction with RT, we have looked at the gene-RT relative risk

$$RR = exp ( X \text{?} )$$

$$\text{?} \text{indicates text missing or illegible when filed}$$

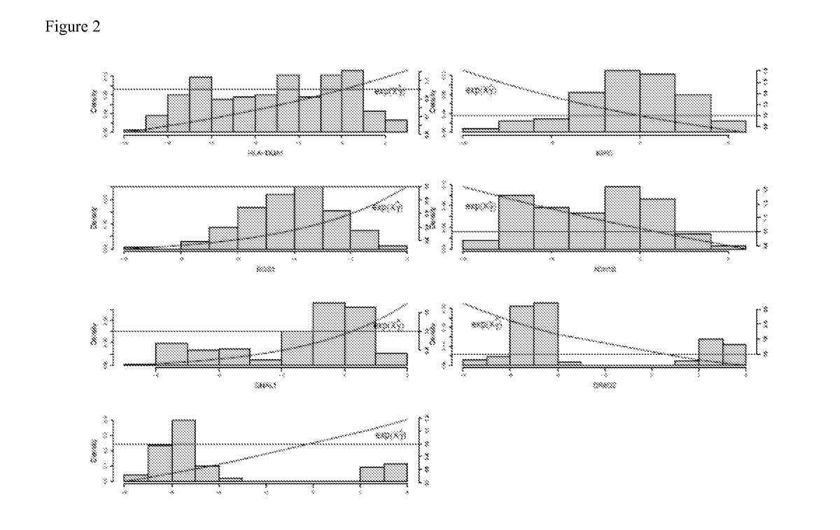

of the I7 genes. Note that these relative risks indicate the conditional influence of each gene, fixing the effects of the others. The seven panels in FIG. 2 show the plots of the gene-RT relative risks (as a function of expression) superimposed on the histogram of each gene expression values in the cohort. The left panels of FIG. 2 shows the relative risks (RR) for HLA-DQA, RGS1, DNALI1 and hCG2023290; all increasing with the expression level. For most patients, genes HLA-DQA, DNALI1 and hCG2023290 interact positively with RT to reduce the risk of LRR. However, for a smaller proportion of women with high gene expression levels, (about 20% for HLA-DQA and hCG2023290 and 31% for DNALI1), the RR is above one indicating an increased risk when radiation is given to such patients. For RGS1, the RR is below one for all samples, thus the marginal effect of this gene is a reduction of the risk of LRR through its association with RT. The three right-most panels of FIG. 2 represent IGKC, ADH1B and OR8G2. They share the same pattern: mostly above one indicating higher risk when these genes are low expressed. Women with high expression levels of these genes (approximately 45%, 25% and 27% for IGKC, ADH1B and OR8G2, respectively) will experience improved LRR-free survival if given radiotherapy.

To evaluate the gene signature 17 as a whole, a cross-validated score index

$${CVSI}_{i} = {\hat{a}}^{( - i )} + \text{?} {\hat{γ}}_{g}^{( - i )} {X}_{i , g}$$

$$\text{?} \text{indicates text missing or illegible when filed}$$

was computed for each woman, following the leave-one-out cross validation scheme described above. The estimated effects [symbol] α⁽⁻ⁱ⁾ of RT were negative for all samples indicating a reduction of the hazard of LRR due to radiation. The mean value of the cross-validated coefficients was

$$\text{?} = - 1.3261$$

$$\text{?} \text{indicates text missing or illegible when filed}$$

corresponding to a decrease of about 26.5% in the risk of LRR. We will refer to the latter as the baseline benefit of RT, which is common to all individuals and independent of the woman's gene expression profile. We considered also the reduced

$${CVSI}_{i} = \text{?} {\hat{γ}}_{g}^{( - i )} {X}_{i , g} . \\ \text{?} \text{indicates text missing or illegible when filed}$$

excluding the baseline, which can be interpreted as the additional individual specific responsiveness to RT. A histogram of the reduced score index CVSIi for all samples is shown in FIG. 3. It is negative for about 95% of the patients, indicating that for most patients in our cohort the 7-genes will interact positively with radiotherapy to further reduce the hazard of LRR. While a small proportion of patients (approximately 5%) have a positive score, indicating a reduced benefit from RT.
Table 2: The seven interaction genes 17. In the first column the AB-specific ID numbers of the seven genes, in the second column the gene symbols, if available. The estimated interaction coefficients and the hazard ratios together with bootstrapped standard errors in parenthesis, are reported in the third and fourth column. A short description of the genes is given in the last column.

TABLE 2

| | Gene | | RR = | |
|---|---|---|---|---|
| AB-ID | Symbol | ( [symbol] ) | exp( [symbol] ) | Description |
| hCG2042724 | HLA- | 0.0099 | 1.0724 | major his [symbol] mpatibility |
| | DQA1 | (0.0440) | (0.0412) | complex, class II, DQ |
| | | | | alpha 1 chr. 6p21.3 |
| hCG1980528.1 | IGKC | −0.0646 | 0.9375 | immunoglobulin kappa |
| | | (0.0426) | (0.0455) | constant chr. arm 2p12 |
| hCG39901.3 | RGSI | 0.2810 | 1.3244 | regulator of G-protein |
| | | (0.1323) | (0.1115) | signalling 1 chr. 1q31 |
| hCG41484.2 | ADH1B | −0.0314 | 0.9691 | alcohol dehydrogenase |
| | | (0.0305) | (0.0325) | IB (class I), beta |
| | | | | polypeptide chr. |
| | | | | 4q21-q23 |
| hCG25678.3 | DNAL1 | 0.3763 | 1.4565 | dynein, axonemal, |
| | | (0.1429) | (0.1130) | light intermediate |
| | | | | polypeptide 1 |
| | | | | chr. arm 1p35.1 |
| hCG2032658 | OR [symbol] G2 | −0.1266 | 0.8811 | olfactory receptor, |
| | | (0.0036) | (0.0699) | family [symbol] , subfamily |
| | | | | G member 2 chr. |
| | | | | 11q24 |
| hCG2023290 | | 0.0452 | 1.0462 | Unknown. chr 7, |
| | | (0.0186) | (0.0179) | |

[symbol]

indicates data missing or illegible when filed

### Internal Validation of the 7-Gene Signature

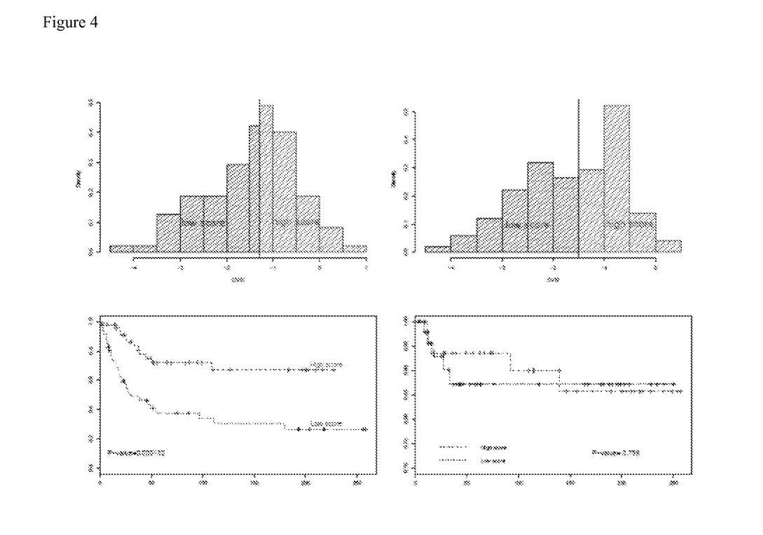

To assess the predictive character of the 7-gene signature, the CVSI was regressed on time to LRR in a simple univariate Cox model. The coefficient of the score was −0.486 (se 0.134, p-value <0.0004) indicating a significant association with the outcome. Another analysis of reliability of the I7-interaction gene signature was based on comparing the relapse-free survival probabilities of women with high vs. low CVSI. First the 195 women were divided into two groups based on receiving RT or not. Within each subgroup (RT and no-RT cohorts) women were further categorized into high and low CVSI classes, by splitting at the median CVSI value. In line with the definition of the CVSI, the two score groups represent women who would benefit most (low CVSI) or least (high CVSI) from radiation. The histograms of the CVSI, with a vertical line at the median, are plotted in FIG. 4 (upper panel): top left for the no-RT and top right for the RT group. The Kaplan-Meier curves for LRR-free survival of the two score groups are shown in FIG. 4. The log-rank test was strongly significant in the no-RT cohort (p-value=10-4). The LRR-free survival probability of women with low CVSI is clearly poorer than for those with high CVSI. Among patients who actually got radiotherapy the difference in LRR-free survival between low and high CVSI was not significant (log-rank test, p-value=0.799).

TABLE 3
The clinical factors associated with LRR-free survival.

| clinical factors | θ | se*(θ) | RR = exp(θ) | se*(RR) |
|---|---|---|---|---|
| RT | −1.3334 | 0.8124 | 0.2634 | 0.2049 |
| Tumor size | 0.5390 | 0.2596 | 1.7160 | 0.3758 |
| # positive nodes | 0.1199 | 0.3513 | 1.1275 | 0.6176 |

Selection of Genes Associated with Distant Metastasis

We applied the double selection procedure with time to distant metastasis (DM) as an outcome. 192 genes were preselected as main effects and 30 as interaction genes, with an overlap of 16 genes. Regressing these candidate genes along with the clinical covariates on time to DM, in a L1-penalized Cox model and applying a 5-fold cross-validation to optimize the penalty weight λopt, 36 main effect genes and two RT-interaction genes: SCGB2A1 and SCGB1D2 were found. Both SCGB2A1 and SCGB1D2 appeared to be positively associated with the risk of DM through their interaction with RT; indicating a reduced DM-free survival probability with increased expression level of these genes. The Lasso estimates of the interaction effects, the corresponding relative risks and the bootstrapped standard errors are reported in Table 4. Tumor size, number of positive lymph nodes and oestrogen receptor status showed a significant association with the DM-free survival, their estimated coefficients and relative risks are given in Table 5.

### The 7-Gene Signature and Distant Metastasis.

We investigated how the 7 interaction genes were associated to the alternative outcome distance metastasis (DM). It is contemplated that these genes interact with RT to reduce the hazard of LLR but can do little to prevent DM in women who have already a diffused disease at the time of treatment. The number of positive lymph nodes was utilized as a proxy for the level of spread of the disease at time of surgery and 11 women with negative lymph nodes were excluded from subsequent analyses. It is further contemplated that the 1-3 nodal group consists of patients with localized tumor and hence within this group, the 17 genes influence the risk of DM through their interaction with RT while for patients with 4+ nodes, these genes have no or little predictive power as this women would have spread tumors. A simple Cox model including the 7 genes and RT was fitted as main effects along with gene-RT interaction terms. For women with 1-3 nodes, the radiotherapy-gene interaction effects for IGKC and RGS1 were significant (p-values 0.04 and 0.001, respectively). In the 4+ nodal group, we found one gene: DNALI1 which influences the risk of DM significantly (p-value 0.025) via its association with RT. An adjustment for tumor size and number of positive nodes lead to a stronger finding, with one significant gene in the 1-3 nodal group (IGKC) and none in the 4+ nodal group. To check whether the expression levels of these genes differ between the two nodal groups we compared the histograms of the expression levels. Only DNALI1 appeared to be differentially expressed in the two groups, with lower expression level in the 1-3 nodes group.

TABLE 4
The two selected genes and the clinical variables.

| AB-ID | Gene Symbol | ( [symbol] ) | RR = exp( [symbol] ) |
|---|---|---|---|
| hCG1731269 | SCGB2A1 | 0.01304 | 1.0131 |
| | | (0.00343) | (0.00337) |
| hCG39846 | SCGB1D2 | 0.0385 | 1.0392 |
| | | (0.00818) | (0.007985) |

[symbol]

indicates data missing or illegible when filed

TABLE 5
The clinical factors associated with DM-free survival.

| clinical factors | θ | se*(θ) | RR = exp(θ) | se*(RR) |
|---|---|---|---|---|
| RT | −0.2334 | 0.1220 | 0.79181 | 0.1586 |
| Tumor size | 0.0306 | 0.0267 | 1.0311 | 0.0287 |
| # positive nodes | 0.0889 | 0.2961 | 2.6874 | 0.4693 |
| ER status | −0.0178 | 0.0771 | 0.9825 | 0.0614 |

### Example 2

### Distribution of the Gene Index Across Groups of Clinical Variables

The two index groups (high and low) could be identified in all relevant clinic-pathological subgroups (tumorsize, malignancy grade, estrogen receptor status, HER2 status, age/menopausal status) (Table 5). Except for estrogen receptor status, there was no significant difference in the distribution of clinico-pathological variables, including nodal status, between the index groups. For all subgroups, except estrogen receptor negative tumors, the distribution of the two index groups was split approximately on the quartile. This indicates that the index is independent of conventional prognostic factors.

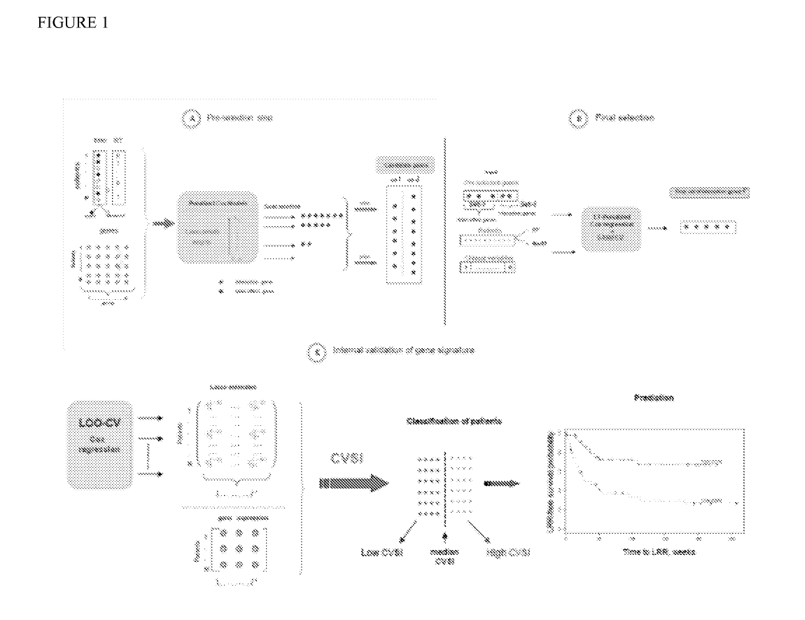

In general, the gene index retained the predictive value across the various clinical parameters. The index was found be predictive regardless of nodal status, when looking at the low index patients (FIG. 1). Nodal status is presently a key parameter in treatment decision process regarding postmastectomy radiotherapy. However, 22% of the patients with an a priori high risk of recurrence (≧4 positive lymph nodes) could be identified as having a high index, and are therefore not expected to gain additional benefit from radiotherapy.

TABLE 5
The table shows the distribution of the two index
groups across various clinically relevant parameters
among 180 lymph node positive patients

| | | | HR (95% CI) |
|---|---|---|---|
| Clinico-pathological | | | low index |
| variables | High index | Low index | PMRT vs. |
| (11 N0 excluded) | N (%) | N (%) | noPMRT |
| Total | 46 (26%) | 134 (74%) | |
| Nodal status | | | |
| 1-3 positive nodes | 28 (29%) | 70 (71%) | 0.16 (0.05; 0.49) |
| ≧4 positive nodes | 18 (22%) | 64 (78%) | 0.21 (0.08; 0.52) |
| Tumor size | | | |
| <2 cm | 11 (20%) | 44 (80%) | 0.11 (0.02; 0.50) |
| 2-5 cm | 30 (30%) | 71 (70%) | 0.22 (0.09; 0.54) |
| ≧5 cm | 5 (21%) | 19 (79%) | 0.42 (0.08; 2.09) |
| Estrogenreceptor status | | | |
| Positive | 43 (33%) | 86 (67%) | 0.13 (0.04; 0.39) |
| Negative | 3 (6%) | 48 (94%) | 0.28 (0.11; 0.72) |
| HER2 status | | | |
| Positive | 7 (15%) | 39 (85%) | 0.30 (0.11; 0.84) |
| Negative | 39 (29%) | 95 (71%) | 0.14 (0.06; 0.37) |
| Menopausal status | | | |
| Pre-menopausal | 21 (25%) | 62 (75%) | 0.29 (0.10; 0.80) |
| Post-menopausal | 25 (26%) | 72 (74%) | 0.13 (0.05; 0.34) |
| Age | | | |
| <50 years | 28 (24%) | 91 (76%) | 0.20 (0.09; 0.45) |
| ≧50 years | 18 (30%) | 43 (70%) | 0.15 (0.03; 0.67) |
| Malignancy grade | | | |
| Grade I | 13 (34%) | 25 (66%) | |
| Grade II | 25 (26%) | 70 (74%) | 0.12 (0.04; 0.35) |
| Grade III | 8 (21%) | 31 (79%) | 0.47 (0.15; 1.49) |

### Example 3

### Validation of Signature in New Preparation Type and on New Platform

### Same Cohort of Patients

In the original cohort, the signature was developed based on RNA extracted from frozen tumour biopsies and gene expression was measured using the Applied Biosystem Human Genome Survey Microarray.

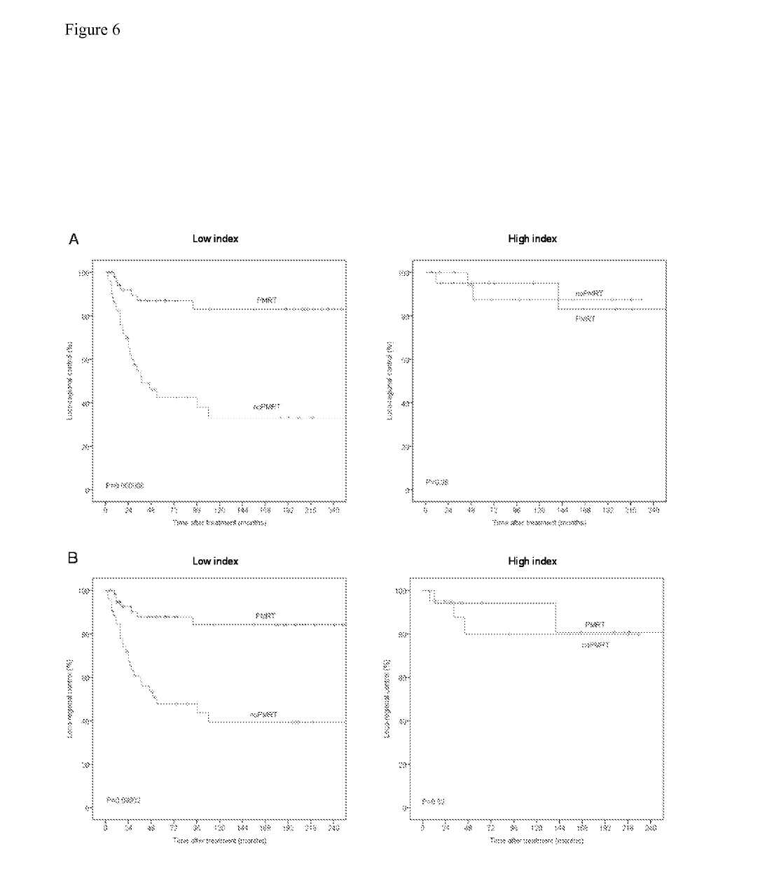

A separate, corresponding part of the same tumour was available as formalin-fixed paraffin-embedded (FFPE) tissue from 158 patients in the original cohort. RNA was extracted from FFPE tissue and converted to cDNA, cDNA was pre-amplified using gene-specific primers, analyzed by qPCR, and normalized to four reference genes as described (Tramm T, Sorensen B S, Overgaard J, Alsner J. Optimal reference genes for normalization of qRT-PCR data from archival formalin fixed, paraffin embedded breast tumors controlling for tumor cell content and decay of mRNA. Diagn Mol Pathol, in press, 2013). Four of the signature genes were analyzed (IGKC, RGS1, DNALI1, ADH1B). All four reference genes and at least 1 of the signature genes could be detected in 150 patients. The predictive impact of the signature was confirmed, as the 75% of the patients with the lowest index had a significant benefit from PMRT whereas the 25% with the highest index had no benefit from PMRT. See FIG. 6.

### Example 4

### Validation of Signature in Independent Patient Cohort

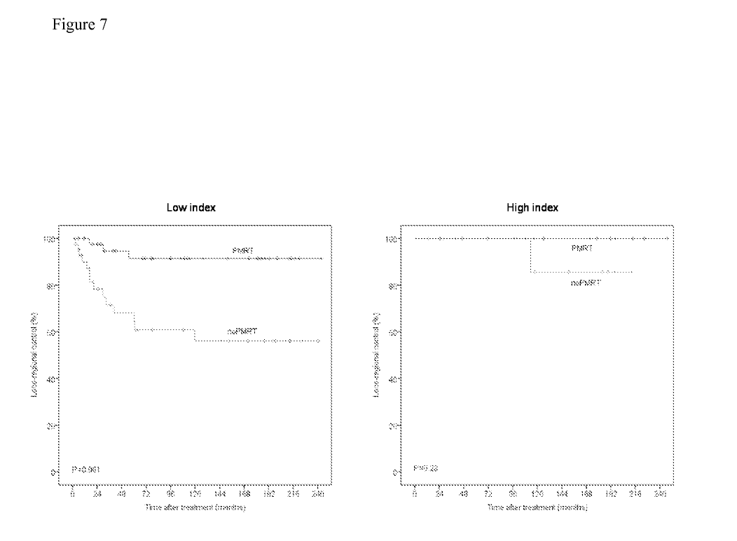

The original cohort consisted of a subset of patients from the Danish Breast Cancer Group 82 b and c cohort (DBCG82bc) where both frozen and FFPE material was available. The independent validation cohort consisted of 931 patients from DBCG82bc where only FFPE material was available and was analyzed using qRT-PCR (as above). All four reference genes could be measured in 871 patients. Of these, all four signature genes (IGKC, RGS1, DNALI1, ADH1B) could be measured in 116 patients. The predictive impact of the signature was confirmed in the independent patient cohort, as the 75% of the patients with the lowest index had a significant benefit from PMRT whereas the 25% with the highest index had no benefit from PMRT. See FIG. 7.

In a series of FFPE samples originating from routinely processed, surgical breast specimens, stored for up to 30 years, optimal genes for normalization was identified (Tramm T, Sørensen BS, Overgaard J, Alsner J. Optimal reference genes for normalization of qRT-PCR data from archival formalin fixed, paraffin embedded breast tumors controlling for tumor cell content and decay of mRNA. Diagn Mol Pathol, in press, 2013). Overall, the half-life of RNA in these samples was 4.6 years. In the same samples, the rate of success in measuring all the 4 signature genes was tested. In the samples that had the same age as the samples from the independent validation cohort (1983-1988), a similar rate of success was observed (17%). In the intermediate age ranges (1989-2005), the success rate was 55%. In recent samples where sufficient amount of RNA is available (2006-2011), the success rate was 100%.

### Example 5

### Statistical Re-Analysis of Genes Predicting Response to Post-Mastectomy Radiotherapy

This example provides data relating to a determination of whether there are additional genes, which can be used in alternative, or together with the 7 genes originally identified, to predict the usefulness of radiotherapy (RT).

Two methods were used: 1) Analysis of a reduced/revised dataset using one-step Lasso with interactions; and 2) Analysis of the reduced dataset using the direct method of Tian, Alizadeh, Gentles and Tibshirani (December 2012).

Validation of the Estimated Index by Survival Curves.

The new gene signature was evaluated exactly as for the original signature: the subjects were divided with the 75% with the lowest index and the 25% with the highest index, and two survival curves were plotted: one for the women who received RT and one for those who did not receive RT. An interaction gene set is considered successful if the two survival curves are significantly different in the first plot, for the 75% lower index, while the two survival curves are not significantly different for the other group of women, with the 25% highest index.

Data:

The data differ from the first analysis as follows:

a) Only probes corresponding to genes with a known name were included. This reduced the number of probes by ca 25%. One of the original seven genes for example, is not part of the new data set.

b) The number of clinical cases is reduced to from 195 to 191 in some runs, after removing 4 cases without histologically verified tumor cell content in the frozen sample.

c) The outcome was redefined in order to follow the statistical principles previously applied to the DBCG82bc cohort. This in particular moved 14 cases from censored to not censored. About 75% of the cases are censored.

d) Tumor size was included as a continuous instead of a categorical covariate

e) HER2 was included as a clinical covariate

f) Malignancy grade was included as a clinical covariate.

Four data sets derived from the above general data were analyzed. The choices are: A) Include or exclude lymph node negative cases (NO); B) Include or exclude the clinical covariates (clin). The four possibilities are denoted as follows: noN0clin: Clinical covariables included, N0 cases excluded (considered as the most relevant data set); N0clin: Both clinical covariables and NO cases included; noN0noclin: Neither clinical variables (except RT) nor N0 cases included; N0noclin: NO cases included, but not clinical covariables.

The noN0clin data set is considered the most relevant, but other data sets were included for completeness of analysis and to provide additional information.

1. Analysis of the Reduced Dataset Using One-Step Lasso with Interactions

Key Statistical Details:

The penalized partial log likelihood was utilized, where we include all gene expressions and all gene expression times RT interactions, and penalized all the coefficients (called beta for the main effects and gamma for the interactions) with only one penalization parameter lambda. We centered the gene expressions and standardized the standard deviation to 1, and then standardized again in the same way the interactions (gene expression times RT). When clinical covariates are present in the models, they are standardized in the same way. R function glmnet is used.

Choice of Lambda.

The results of the Lasso depend on the choice of the parameter lambda. For a large lambda, no interaction genes are selected; for smaller and smaller lambda, more and more interaction genes are selected. We used cross-validation to chose the optimal lambda. However, as the CV curves can be quite flat around the optimal lambda, which means that there is no justification to select the optimal lambda compared to smaller values of lambda, we determined the interaction genes for a series of lamba: starting from the optimal lambda, we reduced the lambda, until the 95% confidence band around the CV curve does not cover anymore the CV score in the optimal lambda. We call this smallest lambda lambda_no_overlap, and the optimal one lambda_opt. All the chosen interaction genes, for a series of values of lambda between lambda_no_overlap and lambda_opt, are equally interesting, and therefore we opted for looking at the UNION of the interaction genes, selected for all these choices of lambda. This gives more interaction genes.

We used 10-fold CV, which is standard. This means that we divide the data in ten equal parts, by partitioning the data into ten parts at random and obtain a set of interaction genes. But if we repeat the same analysis, but using a different random partition, we will get slightly different results. Indeed, because there are many optimization steps in the procedure, the result is known to be very dependent on the random partition. This is why we repeated the analysis for each lambda 100 random times.

Results:

The genes selected for the various sets (in the union of the various lambda and the 100 random repetitions) are provided below. Beside each name is the percentage of runs (for the various selected values of lambda, in the 100 random repetitions), whet was selected. In each of these times, the sign of the coefficient gamma was the same

noN0clin:

| | |
|---|---|
| VANGL1 | 28 |
| C3orf29 | 24 |
| DERP6 | 24 |
| RTCD1 | 23 |
| ZCCHC17 | 15 |
| FLJ37970 | 9 |
| RAF1 | 9 |
| TM2D2 | 3 |
| ZNF22 | 3 |
| MPH0SPH10 | 3 |
| METTL5 | 1 |
| PLA2G5 | 0.1 |

NOnoclin:

| | |
|---|---|
| VANGL1 | 53 |
| RTCD1 | 38 |
| PQLC1 | 37 |
| FAM14B | 36 |
| ZNF616 | 31 |
| LRRC8D | 22 |
| IPLA2(GAMMA) | 20 |
| C3orf29 | 17 |
| FLJ37970 | 16 |
| CLCC1 | 15 |
| sep.01 | 14 |
| PTS | 9 |
| NPB | 6 |
| LOC284739 | 5 |
| LANCL2 | 3 |
| TXNL4A | 3 |
| LPIN2 | 1 |
| FLJ23441 | 1 |
| POLB | 0.2 |
| CAMP | 0.1 |

N0clin:

| | |
|---|---|
| VANGL1 | 58 |
| FAM14B | 36 |
| POLB | 25 |
| TM2D2 | 18 |
| RTCD1 | 12 |
| FLJ37970 | 9 |
| MYO9A | 6 |
| MRPS28 | 3 |
| LOC284739 | 2 |
| PLA2G5 | 2 |
| VDAC3 | 2 |
| FLJ20850 | 1 |
| AKT2 | 1 |
| METTL5 | 1 |
| DBT | 0.2 |

noN0noclin:

| | |
|---|---|
| CLCC1 | 64 |
| DERP6 | 48 |
| RTCD1 | 37 |
| C3orf29 | 37 |
| ANKRD17 | 33 |
| POLB | 28 |
| PQLC1 | 27 |
| PTS | 26 |
| LOC152217 | 20 |
| VANGL1 | 14 |
| FLJ37970 | 11 |
| WIPI1 | 10 |
| LOC284739 | 10 |
| TXNL4A | 9 |
| SCAP1 | 6 |
| RP11-142I17.1 | 5 |
| CLPX | 3 |
| FLJ23441 | 2 |
| EDN2 | 1 |

Smallest Optimal-Equivalent Lambda:

The genes which are selected in the values lambda_no_overlap, which is the largest set, are provided below. The percentage of the gene selected in the 100 runs is given. The most relevant data are those for the noN0clin analysis, of which the most the 7 top ranked genes (highlighted in table below) were used for validation of predictive power.

noN0clin:

| | |
|---|---|
| C3orf29 | 58 |
| RTCD1 | 56 |
| VANGL1 | 51 |
| DERP6 | 45 |
| ZCCHC17 | 31 |
| FLJ37970 | 23 |
| RAF1 | 4 |
| TM2D2 | 2 |
| ZNF22 | 2 |
| MPH0SPH10 | 2 |
| METTL5 | 1 |
| PLA2G5 | 1 |

N0noclin:

| | |
|---|---|
| VANGL1 | 99 |
| PQLC1 | 95 |
| FAM14B | 93 |
| RTCD1 | 91 |
| ZNF616 | 73 |
| LRRC8D | 69 |
| IPLA2(GAMMA) | 60 |
| C3orf29 | 60 |
| FLJ37970 | 55 |
| CLCC1 | 51 |
| sep.01 | 50 |
| PTS | 32 |
| NPB | 19 |
| LOC284739 | 18 |
| LANCL2 | 12 |
| TXNL4A | 12 |
| FLJ23441 | 5 |
| LPIN2 | 4 |
| CAMP | 1 |
| POLB | 0 |

N0clin:

| | |
|---|---|
| VANGL1 | 58 |
| FAM14B | 36 |
| POLB | 25 |
| TM2D2 | 18 |
| RTCD1 | 12 |
| FLJ37970 | 9 |
| MYO9A | 6 |
| MRPS28 | 3 |
| LOC284739 | 2 |
| PLA2G5 | 2 |
| VDAC3 | 2 |
| FLJ20850 | 1 |
| AKT2 | 1 |
| METTL5 | 1 |
| DBT | 0.2 |

noN0noclin:

| | |
|---|---|
| CLCC1 | 92 |
| DERP6 | 77 |
| C3orf29 | 62 |
| POLB | 55 |
| PQLC1 | 52 |
| ANKRD17 | 51 |
| PTS | 50 |
| RTCD1 | 39 |
| LOC152217 | 36 |
| VANGL1 | 25 |
| WIPI1 | 22 |
| FLJ37970 | 16 |
| LOC284739 | 15 |
| TXNL4A | 14 |
| SCAP1 | 13 |
| RP11-142I17.1 | 10 |
| FLJ23441 | 6 |
| CLPX | 4 |
| EDN2 | 2 |

If the lambda_no_overlap analysis is considered, how many times each gene appears in the 400 runs (4 data sets, 100 runs each) can be counted. There were in total 33 genes. The most common genes (out of 400 possible) were:

### VANGL1 (275)

### RTCD1 (226)

C3orf29 (180)

### FAM14B (139)

### PQLC1 (147)

### DERP6 (122).

### POLB (147)

### CLCC1 (143)

### FLJ37970 (130)

Stabilised Random Folds:

We performed also an analysis with random folds generated in such a way that in every fold the number of women with RT was approximately 50%. In this case the same genes as above appeared, but more often, and the genes that appeared very seldom were not selected.

Validation of Predictive Power of New Gene Sets.

As explained before, Kaplan-Meier (KM) estimates are performed for the two groups of women with 75% lowest and 25% highest interaction gene index alpha+sum_{—} {gene in interaction gene set} estimated.gamma_{gene} for the women with and without RT. We then performed log rank tests, to test if the two curves are different. Below we count the number of significant comparisons, in the ten random runs.

P-values for log rank test between KM-curves in the two groups:
group low=index<=75%,
group high=index>75%.

noN0clin: (good)
group low: 61 out of 61 runs are <0.05, the last 39 runs had no selected interaction genes,
group high: all 61>0.05
   This shows that the new set of interaction genes, in particular the ones appearing more often, has the power to identify the women who would benefit most of RT (low index), as shown in the KM plot (FIG. 1). The new set of interactions genes is thus (internally) validated.

N0clin: (not good)
group low: all 100<0.05,
group high: 65 of 100<0.05, but larger pvalues than in the group low

N0noclin: (good)
group low: 99 out of 99<0.05, the last had no interaction genes,
group high: 93 of 99>0.05, 6<0.05

n0N0noclin (good):
group low: 92 out of 92<0.05, the last 8 no interaction genes,
group high: all 92 of 99>0.05
   Except for one data set, the selected genes have a good classification power.

Comparison to the Original Signature Genes in Example 1.

We studied the correlation between the 33 genes in the lambda_no_overlap case (largest number of selected interaction genes) and the previously found genes. In general, the correlation between the 33 genes and the other genes is on average 0.10, with 0.15 being the 75% quartile. This means that correlations of 0.20 or larger are to be considered as special. We have therefore checked if any of the 33 genes has high correlation of 0.20 or more with the original six genes, and we found the following:
1. HLA-DQA1 has high correlation with C3orf29 and RTCD1.
2. IGKC has high correlation with POLB, TM2D2, LOC284739, FLJ37970.
3. RGS1 has high correlation with LOC284739.
4. ADH1B has high correlation with LOC284739, PTS, TXNL4A, LOC152217, MY09A.
5. DNALI1 has high correlation with POLB, MY09A, RTCD1, LOC284739, C3orf29, LANCL2, NPB, LOC152217, SCAP1.
6. OR8G2 has high correlation with AKT2, PQLC1, LPIN2.

The bold underlined interaction genes were selected in noN0clin. In Table 6, we present a correlations between the genes of the original and new signature. It shows that there are many relatively high correlations.

TABLE 6
Correlations (In bold, values that are particularly high, above the 75% quantile)

| | old | | | | | |
|---|---|---|---|---|---|---|
| new | HLA-DQA1 | IGKC | RGS1 | ADH1B | DNALI1 | OR8G2 |
| C3orf29 | 0.20 | −0.06 | 0.10 | −0.07 | 0.07 | −0.09 |
| ZCCHC17 | −0.16 | −0.15 | −0.10 | 0.01 | 0.21 | 0.17 |
| RTCD1 | 0.22 | 0.17 | 0.07 | −0.20 | −0.26 | 0.05 |
| VANGL1 | −0.19 | −0.28 | −0.07 | −0.17 | 0.17 | 0.17 |
| DERP6 | −0.15 | −0.11 | −0.05 | 0.08 | 0.03 | 0.02 |
| FLJ37970 | −0.22 | −0.28 | −0.09 | −0.05 | 0.18 | −0.11 |
| RAF1 | −0.04 | −0.09 | 0.11 | −0.10 | 0.10 | 0.12 |

This finding might justify why the new interaction genes are identified: the current data set is probably sufficiently different from the one originally analyzed to change the composition of the predictive gene signature, but in such a way that the original genes are substituted by other genes (the ones selected in noN0clin), with whom they are positively correlated, at an exceptionally high level.

Next, we analyzed whether the new set of genes allows the prediction of the efficacy of RT in an independent way, with respect to the known clinical variables.

To answer this question, two experiments were performed: 1) In the first one we establish that also the clinical variables can be used to predict which women would benefit most of RT; 2) In the second experiment we compare the new gene signature and the clinical variables as predictors of the efficacy of RT. A Cox model was fit with all clinical covariates (incl. RT) and all the same clinical covariates in interaction with RT. For example, tumor size times RT. The coefficients of the interactions (clinical variables times RT) were estimated and used to construct the index, as we had done before with the interaction genes times RT. The women were divided into two groups (low 75% of this index and high 25%) and in each group two survival curves were estimated, one for the women who actually got RT and one for those who did not. Also here, the KM curves in the first plot were very different (p-value 4.92*10⁻⁸) and the two KM curves in the high index plot were not different (p-value 0.99). This shows that clinical variables can be used to predict the efficacy of RT.

Next, a Cox model was fit with Lasso, including all interactions of all genes times RT and in addition all interactions of the clinical variables times RT. First, Lasso was allowed to select these interactions, if useful for prediction of the LLR. The interactions clinical variables times RT are competed with the interactions gene times RT. Lasso selects the ones which are best for prediction of LLR. The result is that NONE of the clinical variables interactions are selected, but only the new interaction genes (C3orf29, ZCCHC17, RTCD1, VANGL1, DERP6, FLJ37970). This shows that the interactions genes appear to have a stronger predictive power than the clinical variables.

If Lasso is not allowed to select away the interactions between clinical variables and RT, i.e. they are forced into the model, then NONE of the seven new interaction genes are selected. In some few cases these genes are selected instead: SACM1L, VGLL4, ZNF184 (only in 20-30% of the runs). It is thus possible to conclude that the new seven genes seem to be superior to the classical clinical variables, though they also have a predictive power in terms of benefit of RT.

Conclusion:

Given that noN0clin is the appropriate data set, and the positive KM validation of the signature, and the positive correlation of some of these interaction genes with the original ones, the following gene set appears to be most relevant from the new analysis:

C3orf29
ZCCHC17
RTCD1
VANGL1
DERP6
FLJ37970
RAF1

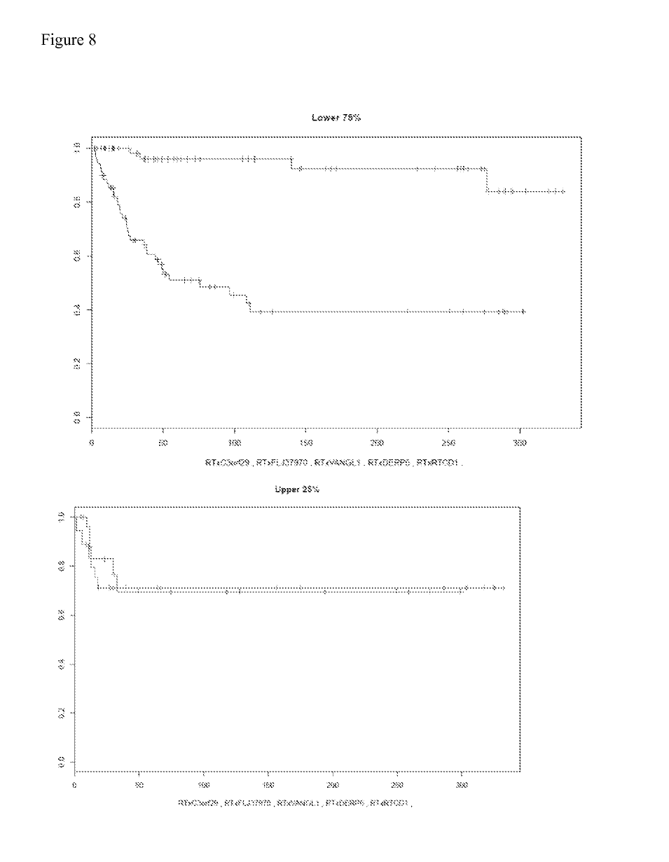

One of the 61 pair of KM plots is provided in FIG. 8. In this specific run, the selected interaction genes were C3orf29, FLJ37970, VANGL1, DERP6, RTCD1. The p-value for the lower 75% group was 2.40*10̂−8 and the p-value for the upper 25% group is 0.9876.

### 2. Analysis of the Reduced Dataset Using the Direct Method of Tian, Alizadeh, Gentles and Tibshirani.

The recently published method of Tian, Alizadeh, Gentles and Tibshirani (2012): A Simple Method for Detecting Interactions between a Treatment and a Large Number of Covariates was used to analyze the data. This Lasso method is designed for use where there are interactions between many covariates and a treatment variable. Here, the idea is to avoid the estimation of the gene expression as main effect, and focus immediately only on interactions. The RT variable is now coded as no RT=−0.5 and RT=0.5. The value 0.5 comes from the randomization of the therapy, which is approximately 50-50. The gene expression values are centered, but not standardized prior to the analysis. The interactions are thus between RT=+/−0.5 and the centered gene expression values. We use the penalized partial log likelihood as previously, and when fitting the model, all variables are standardized within glmnet (the lasso routine) as before.

In this analysis we have only focused on one dataset, noN0clin. The N0 women are excluded and all the clinical data are included in the model, so kept in the model in any case all the time (no Lasso selection on them).

Choice of Lambda:

The results of the Lasso depend on the choice of the parameter lambda. For smaller and smaller values of lambda, more and more genes are selected. We use the lambda_no_overlap as described above, but now we choose the one that is within one standard deviation of the optimal lambda (instead of two, as done in method 1 above). The reason is that now we get quite many more interaction genes, and we wish to reduce the number of interactions.

As before, we used 10-fold CV. It means that we divide the data in ten equal parts, by partitioning the data into ten parts at random and obtain a set of interaction genes. But if we repeat the same analysis, but using a different random partition, we will get slightly different results. Indeed, because there are many optimization steps in the procedure, the result is known to be very dependent on the random partition. This is why we repeated the analysis for each lambda 100 random times.

Results:

The genes which were selected in the values lambda_no_overlap, which is the largest set, for the balanced folds (with %) were as follows:

| | |
|---|---|
| ZDHHC8 | 100 |
| CHCHD6 | 100 |
| MGC40405 | 100 |
| SLC39A7 | 100 |
| LRPAP1 | 100 |
| C1orfl22 | 100 |
| EIF2AK3 | 100 |
| AP1G1 | 100 |
| LYNX1 | 100 |
| FLJ10786 | 100 |
| PPAPDC1B | 99 |
| C9orf44 | 96 |
| RAB11FIP1 | 96 |
| SNRPD2 | 82 |
| TIAM1 | 82 |
| RPSA | 76 |
| LOC441018 | 68 |
| LOC284751 | 50 |
| FRMD4A | 50 |
| HDLBP | 50 |
| CDC42EP2 | 35 |
| PLEK2 | 35 |
| WBP1 | 29 |
| AADAT | 24 |
| COG5 | 24 |
| KIAA1193 | 24 |
| OAZ2 | 24 |
| ANGEL2 | 24 |
| sep.08 | 13 |
| DTYMK | 12 |
| PCYOX1 | 11 |
| POLR2H | 5 |
| COX10 | 5 |
| PROSC | 5 |
| RGS1 | 5 |
| RP3-510O8.5 | 4 |
| SCRN3 | 4 |
| LOC124512 | 4 |
| VNN1 | 4 |
| CHMP4A | 4 |
| POLR1B | 1 |
| C9orf95 | 1 |
| CAMP | 1 |
| LSG1 | 1 |
| GSDMDC1 | 1 |
| C21orf33 | 1 |
| PCGF1 | 1 |
| SLC25A25 | 1 |

This method provides many more interactions genes, and they also appear more regularly in the random folds. It is also noted that RGS1 is selected here (i.e., one of the seven genes). Similar interaction genes are selected, when the folds are not balanced with respect to RT and censoring/event. RGS1 was then selected almost in 50% of the runs. The genes that were found with the first method were not chosen by this second method.

Validation.

As before, Kaplan-Meier estimates are performed for the two groups of women with 75% lowest and 25% highest interaction gene index, for the women with and without RT. We then performed log rank tests, to test if the two curves are different. The number of significant comparisons in 100 random runs are counted below.

P-values for log rank test between KM-curves in the two groups:
group low=index<=75%,
group high=index>75%.

noN0clin: (bad)
group low: all runs have p-value <0.05
group high: also all runs have p-value <0.05

### CONCLUSION

The selected genes by this alternative statistical method do not appear to have any predictive power.

### REFERENCES

[1] Clarke M, Collins R, Darby S, Davies C, Elphinstone P, Evans E et al.; Early Breast Cancer Trialists Collaborative Group (EBCTCG): Effects of radiotherapy and of differences in the extent of surgery for early breast cancer on local recurrence and 15-year survival: an overview of the randomised trials. Lancet 2005; 366: 2087-2106.
[2] National Institutes of Health Consensus Development Conference Statement: adjuvant therapy for breast cancer. Nov. 1-3, 2000, J Natl Cancer Inst 2001; 93: 979-89.
[3] Ragaz J, Olivotto I A, Spinelli J J et al. Locoregional radiation therapy in patients with highrisk breast cancer receiving adjuvant chemotherapy: 20-year results of the British Columbia randomized trial. J Natl Cancer Inst 2005; 97; 116-26 4.
[4] Overgaard M et al. Is the benefit of postmastectomy irradiation limited to patients with four or more positive nodes, as recommended in international consensus reports? A subgroup analysis of the DBCG 82 b&c randomized trials. Radiother Oncol. 2007 March; 82(3):247-53.
[5] Peto R: Highlights from the early breast cancer trialists collaborative group (EBCTCG) 2005-2006 worldwide overview. SABCS 2006; General Session 7: Abstract 40.
[6] Goldhirsch A, Ingle J N, Gelber R D, Coates A S, Thrlimann B, Senn H J; Panel members: Thresholds for therapies: highlights of the St Gallen International Expert Consensus on the primary therapy of early breast cancer 2009. Ann Oncol. 2009 August; 20(8):1319-29.
[7] Kaufmann M, Morrow M, Minckwitz G, Harris J R and the Biedenkopf Expert Panel Members. Locoregional Treatment of Primary Breast Cancer. Consensus Recommendations From an International Expert Panel. Cancer 2010; 116: 1184-1191.
[8] Overgaard M, Hansen P S, Overgaard J, Rose C, Andersson M, Bach F, et al. Postoperative radiotherapy in high-risk premenopausal women with breast cancer who receive adjuvant chemotherapy. Danish Breast Cancer Cooperative Group 82b Trial. N Engl J Med 1997; Oct. 2; 337(14):949-55.
[9] Overgaard M, Jensen M B, Overgaard J, Hansen P S, Rose C, Andersson M, et al. Postoperative radiotherapy in high-risk postmenopausal breast-cancer patients given adjuvant tamoxifen: Danish Breast Cancer Cooperative Group DBCG 82c randomised trial. Lancet 1999; May 15; 353(9165):1641-8.
[10] Nielsen H M et al. Study of Failure Patterns Among High-Risk Breast Cancer Patients With or Without Postmastectomy Radiotherapy in Addition to Adjuvant Systemic Therapy: Long-Term Results From the Danish Breast Cancer Cooperative Group 82 b and c Studies.” J Clin Oncol. 2006 Apr 19.
[11] Kyndi M, Overgaard M, Nielsen H M, Srensen F B, Knudsen H, Overgaard J. High local recurrence risk is not associated with large survival reduction after postmastectomy radiotherapy in high-risk breast cancer: A subgroup analysis of DBCG 82 b&c. Radiother Oncol. 2009 January; 90(1):74-9.
[12] Kyndi M, Srensen F B, Knudsen H, Overgaard M, Nielsen H M, Overgaard J. Estrogen receptor, Progesteron receptor, HER-2, and Response to Postmastectomy in High-Risk Breast Cancer: The Danish Breast Cancer Cooperative Group. J Clin Oncol 2008 March; 26(9): 1419-1426.
[13] Riesterer 0, Milas L, Ang K. Use of Molecular Biomarkers for Predicting the Response to Radiotherapy With or Without Chemotherapy. J Clin Oncol. 2007 September; 25(26): 4075-4083.
[14] Chi J T, Wang Z, Nuyten D S, Rodriguez E H, Schaner M E, Salim A, et al. Gene expression programs in response to hypoxia: cell type specificity and prognostic significance in human cancers. PLoS Med 2006; March; 3(3):e47.
[15] Fisher B, Bryant J Dignam J J et al: Tamoxifen, radiation therapy, or both for prevention of ipsilateral breast tumor recurrence after lumpectomy in women with invasive breast cancers of one centimetre or less. J Clin Oncol 20: 4141-4149, 2002.
[16] Myhre S, Mohammed H, Tramm T, Alsner J, Finak G, Park M, Overgaard J, Borresen-Dale A L, Frigessi A, Srlie T. In silico ascription of gene expression differences to tumor and stromal cells in a model to study impact on breast cancer outcome. PLoS One. 2010 Nov. 19; 5(11):e14002.
[17] Tibshirani R. Regression shrinkage and selection via Lasso. Journal of Royal Statistical Society Series B 1996; 58: 267-395.
[18] Tibshirani R. The Lasso method for variable selection in the Cox model. Statistics in medicine 1997; 16: 385-395.
[19] Van Houwelingen H. C., Bruinsma T., Hart A. A. M., Van′t Veer L. J., & Wessels L. F. A. Cross-validated on microarray gene expression data. Statistics in medicine 2006; 25: 3201-3216.
[20] Verweij P. J. M., van Houwelingen H. C. Cross-validation in survival analysis. Statistics in medecine 1993; 12: 2305-2314.
[21] Debashis P., Bair E., Hastie T., Tibshirani R. Pre-conditioning for feature selection and regression in high-dimensional problems, Annals of Statistics 2008; 36(4), 1595-1618.
[22] Fan J., Lv J. Sure independence screening for ultrahigh dimensional feature space, Journal of the Royal Statistical Society: Series B, 70, 5, 849911, 2008.
[23] Sohn I., Kim J., Jung S-H., Park C. Gradient Lasso for Cox Proportional Hazards Model. Bioinformatics, 2009; Vol. 25, No. 14, 1775-1781.
[24] Gui J., Li H. Penalized Cox regression analysis in the high-dimensional and low-sample size settings, with application to micro-array data. Bioinformatics 2005; 21, 3001-2008.
[25] Park M Y., Hastie T. Ll-Regularization Path Algorithm for Generalized Linear Models. Journal of the Royal Statistical Society B 2007; 69, 659-77.
[26] D. Y. Lin, Goodness-of-fit analysis for the Cox regression model based on a class of parameter estimators. J. Am. Stat. Assoc. 1991; 86: 725-728.
[27] Goeman J J., can de Geer S A, de Kort F., van Houwelingen H C. A global test for groups of genes: testing association with a clinical outcome. Bioinformatics, 2004; 20 (1):93-99.
[28] Efron B., Tibshirani R. (1993). An introduction to the bootstrap. Chapman and Hal, New York.
[29] Noth S, Benecke A. Avoiding inconsistencies over time and tracking difficulties in Applied Biosystems A B1700/Panther probe-to-gene annotations. BMC Bioinformatics 2005; 6: 307.
[30] Chen P C, Tsai E M, Er T K, Chang S J, Chen B H. HLA-DQA1 and -DQB1 allele typing in southern Taiwanese women with breast cancer. Clin. Chem. Lab. Med. 2007; 4 5(5):611-4.
[31] Poulsen T S, Silahtaroglu A N, Gissel C G, Tommerup N, Johnsen H E. Detection of illegitimate rearrangements within the immunoglobulin light chain loci in B cell malignancies using end sequenced probes. Leukemia. 2002; 16(10):2148-55.
[32] The International Multiple Sclerosis Genetics Consortium (IMSGC). I L12A,
MPHOSPH9/CDK2A P1 and RGS1 are novel multiple sclerosis susceptibility loci. Genes Immun. 20101; 11(5):397-405.
[33] Rangel J, Nosrati M, Leong S P, Haqq C, Miller J R 3rd, Sagebiel R W, Kashani-Sabet M. Novel role for RGS1 in melanoma progression. Am J Surg Pathol. 2008; 32(8):1207-12.
[34] Chaudhry M A. Analysis of gene expression in normal and cancer cells exposed to gammaradiation. J Biomed Biotechnol. 2008; 2008:541678.
[35] Visvanathan K, Crum R M, Strickland P T, You X, Ruczinski I, Berndt S I, Alberg A J, Hoffman S C, Comstock G W, Bell D A, Helzlsouer K J. Alcohol dehydrogenase genetic polymorphisms, low-to-moderate alcohol consumption, and risk of breast cancer. Alcohol Clin Exp Res. 2007; 31(3):467-76.
[36] Kawase T, Matsuo K, Hiraki A, Suzuki T, Watanabe M, Iwata H, Tanaka H, Tajima K. Interaction of the effects of alcohol drinking and polymorphisms in alcohol-metabolizing enzymes on the risk of female breast cancer in Japan. J Epidemiol. 2009; 19(5):244-50.
[37] Parris T Z, Danielsson A, Nemes S, Kov'as A, Delle U, Fallenius G, M ollerstr om E, Karlsson P, Helou K. Clinical implications of gene dosage and gene expression patterns in diploid breast carcinoma. Clin Cancer Res. 2010; 16(15):3860-74.
[38] Lacroix M. Significance, detection and markers of disseminated breast cancer cells. Endocr Relat Cancer. 2006; 13(4):1033-67.
[39] Watson M A, Fleming T P. Mammaglobin, a mammary-specific member of the uteroglobin gene family, is overexpressed in human breast cancer. Cancer Res. 1996; 56(4):860-5.
[40] Ouellette R J, Richard D, Ma\ddot{}icas E. R T-PCR for mammaglobin genes, MGB1 and MGB2, identifies breast cancer micrometastases in sentinel lymph nodes. Am J Clin Pathol. 2004; 121(5):637-43.
[41] Tassi R A, Calza S, Ravaggi A, Bignotti E, Odicino F E, Tognon G, Donzelli C, Falchetti M, Rossi E, Todeschini P, Romani C, Bandiera E, Zanotti L, Pecorelli S, Santin A D. Mammaglobin B is an independent prognostic marker in epithelial ovarian cancer and its expression is associated with reduced risk of disease recurrence. BMC Cancer. 2009; 9:253.
[42] Bernstein J L, Godbold J H, Raptis G, Watson M A, Levinson B, Aaronson S A, Fleming T P. Identification of mammaglobin as a novel serum marker for breast cancer. Clin Cancer Res. 2005; 11(18): 6528-35.
[43] Lehrer R I, Xu G, Abduragimov A, Dinh N N, Qu X D, Martin D, Glasgow B J. Lipophilin, a novel heterodimeric protein of human tears. FEBS Lett. 1998 Aug. 7; 432(3):163-7.
[44] Plasman P O, Herchuelz A. Regulation of Na+/Ca2+ exchange in the rat pancreatic B cell. Biochem J. 1992 Jul. 1; 285 (Pt 1):123-7.
[45] Sorlie T., Perou C. M., Fan C., Geisler S., Aas T., Nobel A., Anker G., Akslen L. A., Botstein D., Borresen-Dale A. L, Lonning P. E. Gene expression profiles do not consistently predict the clinical treatment response in locally advanced breast cancer. Molecular Cancer Therapeutics 5 (11), 2914.2918, 2006. et al., Mol Cancer Therapeutics 5 2006
[46] Weichselbaum R R, Ishwaran H, Yoon T, Nuyten D S, Baker S W, Khodarev N, Su A W, Shaikh A Y, Roach P, Kreike B, Roizman B, Bergh J, Pawitan Y, van de Vijver M J, Minn A J. An interferon-related gene signature for DNA damage resistance is a predictive marker for chemotherapy and radiation for breast cancer. Proc Natl Acad Sci USA. 2008 Nov. 25; 105(47):18490-5. Epub 2008 Nov. 10.
[47] Nuyten D S, Kreike B, Hart A A, Chi J T, Sneddon J B, Wessels L F, Peterse H J, Bartelink H, Brown P O, Chang H Y, van de Vijver M J. Predicting a local recurrence after breast-conserving therapy by gene expression profiling. Breast Cancer Res. 2006; 8(5):R62.
[48] Fan C, Prat A, Parker J S, Liu Y, Carey L A, Troester M A, Perou C M. Building prognostic models for breast cancer patients using clinical variables and hundreds of gene expression signatures. BMC Med Genomics. 2011; 4:3.
[49] Rodriguez A A, Makris A, Wu M F, Rimawi M, Froehlich A, Dave B, Hilsenbeck S G, Chamness G C, Lewis M T, Dobrolecki L E, Jain D, Sahoo S, Osborne C K, Chang J C. DNA repair signature is associated with anthracycline response in triple negative breast cancer patients. Breast Cancer Res Treat. 2010; 123(1):189-96.
[50] Bonnefoi H, Potti A, Delorenzi M, Mauriac L, Campone M, Tubiana-Hulin M, Petit T,
Rouanet P, Jassem J, Blot E, Becette V, Farmer P, Andr S, Acharya C R, Mukherjee S, Cameron
D, Bergh J, Nevins J R, Iggo R D. Validation of gene signatures that predict the response of breast cancer to neoadjuvant chemotherapy: a substudy of the EORTC 10994/BIG 00-01 clinical trial. Lancet Oncol. 2007; 8(12):1071-8.
[51] Chang J C, Wooten E C, Tsimelzon A, Hilsenbeck S G, Gutierrez M C, Elledge R, Mohsin S, Osborne C K, Chamness G C, Allred D C, O'Connell P. Gene expression profiling for the prediction of therapeutic response to docetaxel in patients with breast cancer. Lancet. 2003; 362(9381):362-9.
[52] Chang J C, Wooten E C, Tsimelzon A, Hilsenbeck S G, Gutierrez M C, Tham Y L, Kalidas M, Elledge R, Mohsin S, Osborne C K, Chamness G C, Allred D C, Lewis M T, Wong H, O'Connell P. Patterns of resistance and incomplete response to docetaxel by gene expression profiling in breast cancer patients. J Clin Oncol. 2005; 23(6):1169-77.
[53] Weichselbaum R R, Ishwaran H, Yoon T, Nuyten D S, Baker S W, Khodarev N, Su A W, Shaikh A Y, Roach P, Kreike B, Roizman B, Bergh J, Pawitan Y, van de Vijver M J, Minn A J. An interferon-related gene signature for DNA damage resistance is a predictive marker for chemotherapy and radiation for breast cancer. Proc Natl Acad Sci USA. 2008; 105(47):18490-5.
[54] Piening B D, Wang P, Subramanian A, Paulovich A G. A radiation-derived gene expression signature predicts clinical outcome for breast cancer patients. Radiat Res. 2009; 171(2):141-54.
[55] Coutant C, Rouzier R, Qi Y, Lehmann-Che J, Bianchini G, Iwamoto T, Hortobagyi G N, Symmans F, Uzan S, Andre F, de The H, Pusztai L. Distinct p53 gene signatures are needed to predict prognosis and response to chemotherapy in E R-positive and E R-negative breast cancers. Clin Cancer Res. 2011 Jan. 19.

1. A method of providing a prognosis for a subject with breast cancer, selecting a subject with breast cancer for treatment with a particular therapy, determining an increased risk of local recurrence in a subject with breast cancer, or determining an increased risk of distant metastasis in a subject with breast cancer comprising:
a) detecting the level of expression of one or more genes selected from the group consisting of HLA-DQA, RGS1, DNALI1, IGKC, ADH1B, hCG2023290, OR8G2, C3orf29, ZCCHC17, RTCD1, VANGL1, DERP6, F1137970, and RAFT in a sample from a subject diagnosed with breast cancer; and
b) comparing the expression of said one or more genes with a reference expression level for said one or more genes, wherein an altered level of expression relative to said reference provides an indication selected from the group consisting of the likelihood of disease free survival, the likelihood of local recurrence, the likelihood of distant metastasis, and an indication that the subject is a candidate for treatment with a particular therapy.

2. The method of claim 1, wherein said detecting the level of expression of one or more genes comprises determining an expression profile for one or more genes selected from the group consisting of HLA-DQA, RGS1, DNALI1, IGKC, ADH1B, hCG2023290, and OR8G2.

3. The method of claim 2, wherein an increased level of expression of HLA-DQA, RGS1, DNALI1 and a decreased level of expression of IGKC, ADH1B and OR8G2 is associated with an increased risk of local recurrence of breast cancer.

4. The method of claim 1, wherein said detecting the level of expression of one or more genes comprises determining an expression profile for one or more genes selected from the group consisting of C3orf29, ZCCHC17, RTCD1, VANGL1, DERP6, FLJ37970, and RAF1.

5. The method of claim 4, wherein an increased level of expression of one or more of genes selected from the group consisting of C3orf29, ZCCHC17, RTCD1, VANGL1, DERP6, FLJ37970, and RAF1 is associated with an increased risk of local recurrence of breast cancer.

6. The method of claim 1, further comprising the step of determining a treatment course of action based on said level of expression.

7. The method of claim 6, wherein said treatment course of action is administration of post mastectomy radiation.

8. The method of claim 6, wherein said treatment course of action is based on a CSVI score calculated from said expression levels, wherein said
$${CVSI}_{i} = {\hat{a}}^{( - i )} + \text{?} {\hat{γ}}_{g}^{( - i )} {X}_{i , g} . \\ \text{?} \text{indicates text missing or illegible when filed}$$

9. The method of claim 8, wherein a negative CVSI score is indicative of a positive response to said post mastectomy radiation and a positive score is indicative of a negative response to said post mastectomy radiation.

10. The method of claim 7, wherein decreased expression of HLA-DQA, RGS1, DNALI1 and hCG2023290 and increased expression of IGKC, ADH1B and OR8G2 is associated with an increased benefit of radiation therapy.

11. The method of claim 1, wherein altered expression of one or more genes selected from the group consisting of IGKC, RGS1 and DNALI1 is associated with increased risk of distant metastasis in said subject.

12. (canceled)

13. The method of claim 1, wherein said detecting comprises contacting a sample from said subject with at least informative reagent specific for said one or more genes.

14. The method of claim 1, wherein said detecting an expression level comprises detecting the level of nucleic acid of said genes in said sample.

15. The method of claim 14, wherein said detecting the level of nucleic acid of said genes in said sample comprises detecting the level of mRNA of said genes in said sample.

16. The method of claim 15, wherein said detecting the level of expression of said genes comprises a detection technique selected from the group consisting of microarray analysis, reverse transcriptase PCR, quantitative reverse transcriptase PCR, and hybridization analysis.

17. The method of claim 1, wherein said detecting an expression level comprises detecting the level of polypeptide expression from said genes in said sample.

18. A method of characterizing breast cancer in a subject diagnosed with breast cancer, comprising:
a) measuring the level of expression of one of more genes selected from the group consisting of SCGB2A1 and/or SCGB1D2 in a sample from a subject diagnosed with breast cancer; and
b) characterizing breast cancer based on said level of expression.

19. The method of claim 18, wherein said characterizing comprises determining risk of distant metastasis in said subject.

20. The method of claim 19, wherein an increased level of expression of said genes is associated with an increased risk of distant metastasis in said subject.

21-31. (canceled)

32. A kit for characterizing breast cancer in a subject, said kit comprising informative reagents useful, sufficient, or necessary for detecting and/or characterizing level, presence, or frequency of expression of one or more genes selected from the group consisting of HLA-DQA, RGS1, DNALI1, IGKC, ADH1B, hCG2023290, OR8G2, C3orf29, ZCCHC17, RTCD1, VANGL1, DERP6, F1137970, RAFT, SCGB2A1 and SCGB1D2.

33. (canceled)
